(19) United States
(12) Patent Application Publication
(10) Pub. No.: US 2023/0285296 A1
(43) Pub. Date: Sep. 14, 2023

(54) LAYER-BY-LAYER COATED NANOLIPOSOME FOR ORAL DELIVERY OF INSULIN

(71) Applicant: NANYANG TECHNOLOGICAL UNIVERSITY, Singapore (SG)

(72) Inventors: Subramanian VENKATRAMAN, Singapore (SG); Yingying HUANG, Singapore (SG); Yiming ZHANG, Singapore (SG); Minru Gordon XIONG, Singapore (SG); Bernhard Otto BOEHM, Singapore (SG); Yusuf ALI, Singapore (SG)

(21) Appl. No.: 18/006,138

(22) PCT Filed: Jul. 23, 2021
(86) PCT No.: PCT/SG2021/050434

§ 371 Date: Jan. 19, 2023

(30) Foreign Application Priority Data
Jul. 24, 2020 (SG) 10202007118P

(51) Int. Cl. A61K 9/127; A61K 47/24; A61K 38/28; A61P 3/10

(52) U.S. Cl. CPC A61K 9/1271; A61K 9/1277; A61K 38/28; A61K 47/24; A61P 3/10

(57) ABSTRACT

A multilayered liposome disclosed herein includes a liposome core defined by a lipid layer, and five or more coating layers surrounding the lipid layer, wherein the five or more coating layers include more than one positively charged polymeric layer and more than one negatively charged drug layer, wherein the more than one positively polymeric layer and the more than one negatively charged drug layer are deposited in an alternating manner, wherein one of the more than one positively charged polymeric layer is formed as an outmost coating layer, and wherein each of the more than one negatively charged drug layer includes insulin, an insulin-like factor, a growth factor, or a hormonal peptide. In one embodiment, an anion liposome core (HSPC/DPPG) is coated via a layer-by-layer approach with multilayers of oppositely charged insulin and chitosan layers.

### CROSS-REFERENCE TO RELATED APPLICATION

This application claims the benefit of priority of Singapore Patent Application No. 10202007118P, filed 24 Jul. 2020, the content of it being hereby incorporated by reference in its entirety for all purposes.

### TECHNICAL FIELD

The present disclosure relates to a multilayered liposome and a method of producing the multilayered liposome. The present disclosure also relates to uses of the multilayered liposome, including its use for oral delivery of insulin.

### BACKGROUND

Mimicking the physiological route taken by pancreatic insulin may be considered as a desired goal of insulin therapy in people with diabetes. However, delivery of protein drugs, e.g. via an oral route, tends to be challenging because of the chemical and physical barriers in the gastrointestinal (GI) tract. The acidic pH of the stomach and the hydrolytic enzymes in the GI tract may degrade any large molecular weight proteins approaching the intestinal surface, lowering the bioavailability of orally administered drugs, such as insulin. Furthermore, the gaps between adjacent epithelial cells are sealed by tight junctions which limit permeation of drugs to only small hydrophobic molecules (less than 700 Da) and/or even smaller sized hydrophilic molecules (less than 200 Da). Large molecular weight protein drugs tend to have no chance of crossing the epithelial cell barrier even if they survived the harsh conditions of the stomach.

Current efforts for delivery of insulin may be centred around the use of permeation enhancers and insulin analogues. In one reported example, a liposome-based Hepatic-Directed Vesicle (HDV) technology appears to be a nanotechnology that reached clinical phase III trials in the field of oral insulin delivery. The technology utilizes a liver targeting moiety to alter the surface of the nanoliposome (less than 150 nm). By targeting the hepatocytes, a much lower dose of insulin is likely required to control glycemia. The HDV technology seems to be successful at stimulating the liver’s involvement in hepatic glucose uptake and appears useful at preventing hypoglycaemia events, a major drawback of current (injected) insulin regimens. However, the drug loading appears limited with only 1% drug encapsulation by the nanoliposome.

There is thus a need to provide for a solution that addresses one or more of the limitations mentioned above. The solution should at least provide for a carrier of a drug, e.g. insulin, to be orally administered.

### SUMMARY

In a first aspect, there is provided for a multilayered liposome including:
a liposome core defined by a lipid layer; and
five or more coating layers surrounding the lipid layer,
wherein the five or more coating layers include more than one positively charged polymeric layer and more than one negatively charged drug layer,
wherein the more than one positively charged polymeric layer and the more than one negatively charged drug layer are deposited in an alternating manner,
wherein one of the more than one positively charged polymeric layer is formed as an outermost coating layer, and
wherein each of the more than one negatively charged drug layer includes insulin, an insulin-like factor, a growth factor, or a hormonal peptide.

In another aspect, there is provided for use of the multilayered liposome described according to various embodiments of the first aspect in the manufacture of a medicament for the treatment of diabetes mellitus.

In another aspect, there is provided a method of treating diabetes mellitus, the method includes orally administering the multilayered liposome described according to various embodiments of the first aspect.

In another aspect, there is provided a method of producing the multilayered liposome described according to various embodiments of the first aspect, wherein the method includes:
providing liposomes each having a liposome core defined by a lipid layer;
forming one negatively charged drug layer or one positively charged polymeric layer on the liposome core;
depositing one positively charged polymeric layer on the formed negatively charged drug layer or one negatively charged drug layer on the formed positively charged polymeric layer;
repeating the deposition of one negatively charged drug layer on the positively charged polymeric layer earlier deposited or one positively charged polymeric layer on the negatively charged drug layer earlier deposited so as to have
   (i) five or more coating layers surrounding the lipid layer, and
   (ii) the more than one positively charged polymeric layer and the more than one negatively charged drug layer deposited in an alternating manner,
wherein one of the more than one positively charged polymeric layer is formed as an outermost coating layer, and
wherein each of the more than one negatively charged drug layer includes insulin, an insulin-like factor, a growth factor, or a hormonal peptide.

In another aspect, there is provided for a multilayered liposome including:
a liposome core defined by a lipid layer;
five or more coating layers surrounding the lipid layer;
an outermost coating layer which is positively charged;
wherein the five or more coating layers include more than one negatively charged polymeric layer and more than one positively charged drug layer,
wherein the more than one negatively charged polymeric layer and the more than one positively charged drug layer are deposited in an alternating manner; and
wherein each of the more than one positively charged drug layer includes insulin, an insulin-like factor, a growth factor, or a hormonal peptide.

In another aspect, there is provided for a method of producing the multilayered liposome described according to various embodiments of the above aspect, the method includes:
providing liposomes each having a liposome core defined by a lipid layer;
forming one positively charged drug layer or one negatively charged polymeric layer on the liposome core;
depositing one negatively charged polymeric layer on the formed positively charged drug layer or one positively charged drug layer on the formed negatively charged polymeric layer;
repeating the deposition of one positively charged drug layer on the negatively charged polymeric layer earlier deposited or one negatively charged polymeric layer on the positively charged drug layer earlier deposited so as to have
   (i) five or more coating layers surrounding the lipid layer, and
   (ii) the more than one negatively charged polymeric layer and the more than one positively charged drug layer deposited in an alternating manner,
forming an outermost coating layer which is positively charged, and
wherein each of the more than one positively charged drug layer comprises insulin, an insulin-like factor, a growth factor, or a hormonal peptide.

### BRIEF DESCRIPTION OF THE DRAWINGS

The drawings are not necessarily to scale, emphasis instead generally being placed upon illustrating the principles of the present disclosure. In the following description, various embodiments of the present disclosure are described with reference to the following drawings, in which:

FIG. 1 is a schematic drawing of Layer-by-Layer (LbL) coated liposome and the proposed molecular interaction between insulin and 3 different molecular weights of chitosan.

FIG. 2A depicts the dynamic light scattering (DLS) characterization of size of the LbL coated liposome fabricated using alternating layers of chitosan and insulin. Effect of increasing coating layers of insulin and different molecular weights of chitosan (chitosan 15, chitosan 190-50, and chitosan 310-190, from top to bottom image, respectively) on hydrodynamic size are plotted.

FIG. 2B depicts the zeta potential measurements of the LbL coated liposome fabricated using alternating layers of chitosan and insulin. Effect of increasing coating layers of insulin and different molecular weights of chitosan (chitosan 15, chitosan 190-50, and chitosan 310-190, from top to bottom image, respectively) on zeta potential are plotted.

FIG. 3A demonstrates for the effect of increasing coating layers on insulin loading. FIG. 3A is a comparison between insulin loading of liposomes with 3, 5, and 11 alternating layers of chitosan 310-190 and insulin, and uncoated insulin-loaded HSPC/DPPG liposome core (L0). The data are represented by mean ± standard deviation (n = 3, *P < 0.05).

FIG. 3B demonstrates for the effect of polymer length on insulin loading. FIG. 3B is a comparison between insulin loading of liposomes with 11 alternating layers of insulin and chitosan, fabricated with chitosan of different molecular weights. The data are represented by mean ± standard deviation (n = 3, *P < 0.05).

FIG. 4A demonstrates for the release profile of the LbL coated liposome in simulated gastric fluid (SGF) pH 1.2 and simulated intestinal fluid SGF of pH 6.8. Specifically, FIG. 4A demonstrates for the release profile of 11-layers LbL coated liposome in SGF pH 1.2 and SIF pH 6.8.

FIG. 4B compares the release profiles between 3 and 5 layer LbL coated liposome and uncoated liposome (L0) in PBS pH 7.4.

FIG. 4C demonstrates for the release and stability of the LbL coated liposome in terms of hydrodynamic size in PBS pH 7.4.

FIG. 4D demonstrates for zeta potential of 3 and 5 layer LbL coated liposome and uncoated liposome (L0) in PBS pH 7.4.

FIG. 5A demonstrates for cellular uptake and transport of LbL coated liposome by Caco-2 cells. The top image in FIG. 5A shows various confocal microscopy imaging of Caco-2 monolayers at different z-positions. Red represents immunostaining for Claudin-1 (see 1^{st} column of images). Green represents LbL coated liposome fluorescently tagged with coumarin-6 in the liposome lipid bilayer (see 2^{nd} column of images). Blue represents DAPI staining (see 3^{rd} column of images). The last column shows merged images of red, green and blue staining results. The bottom image in FIG. 5A is a 3D rendering of the stack of confocal images with the orientation representing apical to basal. Inset represents schematic drawing illustrating the position of the fluorescence tag in the lipid bilayer of the liposome core. Scale denotes for 50 µm.

FIG. 5B demonstrates for cumulative transport of insulin across Caco-2 cells over 4 hours. Amount of insulin measured by ELISA.

FIG. 5C demonstrates for TEER measurement during the transport study.

FIG. 5D demonstrates for Alamar blue assay of Caco-2 cells immediately performed after the transport study.

FIG. 6A demonstrates for differentiation of 3T3 L1-MBX fibroblast into adipocytes for the bioactivity study of transported insulin. Specifically, FIG. 6A shows morphology of the 3T3 L1-MBX fibroblast after 0 days of differentiation. Scale bar denotes for 100 µm.

FIG. 6B demonstrates for differentiation of 3T3 L1-MBX fibroblast into adipocytes for the bioactivity study of transported insulin. Specifically, FIG. 6B shows morphology of the 3T3 L1-MBX fibroblast after 12 days of differentiation. Scale bar denotes for 100 µm.

FIG. 6C demonstrates for differentiation of 3T3 L1-MBX fibroblast into adipocytes for the bioactivity study of transported insulin. Specifically, FIG. 6C shows morphology of the 3T3 L1-MBX fibroblast after 14 days of differentiation. Scale bar denotes for 100 µm.

FIG. 6D demonstrates for differentiation of 3T3 L1-MBX fibroblast into adipocytes for the bioactivity study of transported insulin. Specifically, FIG. 6D shows morphology of the 3T3 L1-MBX fibroblast after 25 days of differentiation. Scale bar denotes for 100 µm.

FIG. 6E shows oil red staining of matured 3T3 L1-MBX adipocytes.

FIG. 6F shows a magnified image of FIG. 6E.

FIG. 6G depicts glucose uptake insulin response curve of transported insulin performed on matured 3T3 L1-MBX adipocytes. The data are represented by mean ± standard deviation (n = 3, *P < 0.05).

FIG. 6H depicts glucose uptake of transported insulin performed on matured 3T3 L1-MBX adipocytes. The data are represented by mean ± standard deviation (n = 3, *P < 0.05).

FIG. 7 demonstrates for lyophilisation of LbL coated liposome with varying concentrations of cryoprotectants, sucrose and trehalose.

FIG. 8 demonstrates for in vivo pharmacokinetics study of insulin loaded in LbL-coated liposome orally administered to Wistar rats.

FIG. 9A depicts the hydrodynamic sizes for LbL coated liposome having various number of coated layers. Chitosan 310-190 and a model protein of Bovine Serum Albumin (BSA) were used. Effect of increasing number of coating layers on hydrodynamic size is plotted.

FIG. 9B depicts the zeta potentials for LbL coated liposome having various number of coated layers. Chitosan 310-190 and a model protein of BSA were used. Effect of increasing number of coating layers on zeta potential is plotted.

FIG. 10A demonstrates for change in hydrodynamic size of the LbL coated liposome (11 layers, chitosan 310-190 kDa/insulin) in SGF pH 1.2 over time.

FIG. 10B demonstrates for change in zeta potential of the LbL coated liposome (11 layers, chitosan 310-190 kDa/insulin) in SGF pH 1.2 over time.

FIG. 11A demonstrates for the effect of increasing coating layers on LbL coated liposome release profile in SIF pH 6.8 at 37° C. Specifically, FIG. 11A compares the release profile of 3, 5, and 11 layers of chitosan 310-190/insulin coated LbL liposome in SIF pH 6.8 at 37° C. L3, L5 and L11 denote for total of 3, 5, 11 layers, respectively.

FIG. 11B demonstrates for the effect of polymer length on LbL coated liposome release profile in SIF pH 6.8 at 37° C. Specifically, FIG. 11B compares the release profile of 11 layered LbL liposomes fabricated with insulin, and chitosan of different molecular weights, in SIF pH 6.8 at 37° C.

FIG. 11C depicts the particle size of the liposomes of FIG. 11A. DLS was used to measure the particle size. The data is represented by mean ± standard deviation (n = 3, *P < 0.05).

FIG. 11D depicts the particle size of the liposomes of FIG. 11B. DLS was used to measure the particle size. The data is represented by mean ± standard deviation (n = 3, *P < 0.05).

FIG. 12 depicts release profile of LbL liposomes in PBS pH 7.4 at 37° C. over 12 weeks, specifically comparing between release of 3 (L3) and 5 (L5) layers of chitosan 310-190/insulin coated LbL liposome and insulin loaded HSPC/DPPG liposome core (L0).

FIG. 13 depicts the size (bar graph) and polydispersity index (PDI) (trend line) measured after each layer of coating.

FIG. 14A demonstrates for release of LbL-liposomes after 3 and 5 layers of coating with oppositely charged chitosan and insulin in PBS 7.4 at 37° C.

FIG. 14B demonstrates for loading of 3 and 5 layers coated LbL-liposomes assessed by the ratio between cumulatively released weight of insulin and lyophilized weight of insulin loaded particle.

FIG. 14C demonstrates for fold of increased loading calculated based on the ratio between the loading of LbL-liposomes and loading of uncoated HSPC DPPG liposome.

FIG. 15A demonstrates for transport of insulin loaded LbL-liposomes across caco-2 cells with Eudragit S100 coated at the outermost layer.

FIG. 15B demonstrates for TEER readings measured during transport study.

FIG. 15C demonstrates for cellular uptake of LbL-liposomes imaged by fluorescence microscopy. Green channel denotes AF488 anti-insulin antibody staining of insulin. Red channel denotes AF568 anti-claudin-1 antibody staining of claudin-1 of the tight junction. Scale bar denotes for 100 µm.

FIG. 16A demonstrates for stability of LbL coated DPPG/HSPC liposomes in SGF by measuring the change in size following 4 weeks of incubation at 37° C. in SGF.

FIG. 16B demonstrates for stability of LbL coated DPPG/HSPC liposomes in PBS by measuring the change in size following 4 weeks of incubation at 37° C. in PBS.

FIG. 16C demonstrates for stability of LbL coated DPPG/HSPC liposomes in SGF by measuring the change in zeta potential following 4 weeks of incubation at 37° C. in SGF.

FIG. 16D demonstrates for stability of LbL coated DPPG/HSPC liposomes in PBS by measuring the change in zeta potential following 4 weeks of incubation at 37° C. in PBS.

FIG. 16E demonstrates for stability of LbL coated DPPG/HSPC liposomes in SGF by measuring the change in PDI following 4 weeks of incubation at 37° C. in SGF.

FIG. 16F demonstrates for stability of LbL coated DPPG/HSPC liposomes in PBS by measuring the change in PDI following 4 weeks of incubation at 37° C. in PBS.

FIG. 17A is a plot of the total surface area of the LbL coated liposomes using 15 kDa chitosan against the number of layers coated.

FIG. 17B is a plot of the estimated plasma insulin coated on the LbL coated liposomes using 15 kDa chitosan against the number of layers coated.

FIG. 18A is a plot of the total surface area of the LbL coated liposomes using 310-190 kDa chitosan against the number of layers coated.

FIG. 18B is a plot of the estimated plasma insulin coated on the LbL coated liposomes using 310-190 kDa chitosan against the number of layers coated.

### DETAILED DESCRIPTION

The following detailed description refers to the accompanying drawings that show, by way of illustration, specific details and embodiments in which the present disclosure may be put in practice.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The present disclosure relates to a multilayered liposome for a drug to be orally administered. The “multilayered liposome” is termed herein a “drug carrier” and “carrier”, as the multilayered liposome is capable of delivering a drug. Interchangeably, the multilayered liposome may be termed herein a “multilayered nanoliposome”, a “multilayered particle”, and a “multilayered nanoparticle”. For brevity, the present multilayered liposome may be termed herein “liposome” or “nanoliposome”, or abbreviated as “Layer-by-layer (LbL) liposome”.

Details of various embodiments of the present multilayered liposome and advantages associated with the various embodiments are now described below. Advantages described in the example section of the present disclosure are not reiterated for brevity.

In the present disclosure, in a first aspect, there is provided a multilayered liposome that may include a liposome core defined by a lipid layer, and five or more coating layers surrounding the lipid layer. The five or more coating layers may include more than one positively charged polymeric layer and more than one negatively charged drug layer, wherein the more than one positively charged polymeric layer and the more than one negatively charged drug layer may be deposited in an alternating manner, wherein one of the more than one positively charged polymeric layer may be formed as an outermost coating layer, and wherein each of the more than one negatively charged drug layer may include a drug. The drug may include insulin, an insulin-like factor, a growth factor, or a hormonal peptide. Advantageously, as the drug is encapsulated between the layers and internal to the outermost layer, the drug does not get compromised as it migrates through the harsh environment of the GI tract. Also, as the drug is present in the layers, the drug is able to permeate out of the liposome easily (at its target destination) as compared to a drug in the liposome core. Further advantageously, a positively charged outermost coating layer formed by the positively charged polymeric layer may enhance interaction with negatively charged mucin and cell surface, which increases the retention time and absorption of the present multilayered nanoliposome.

In various embodiments, the lipid layer may be positively charged or negatively charged. The lipid layer may be or may include hydrogenated soybean phosphatidylcholine (HSPC), 1,2-dipalmitoyl-sn-glycero-3-phosphoglycerol (DPPG), and/or 1,2-dioleoyl-3-trimethylammoniumpropane. In certain non-limiting embodiments, the lipid layer may be negatively charged and includes hydrogenated soybean phosphatidylcholine and 1,2-dipalmitoyl-sn-glycero-3-phosphoglycerol in a molar ratio of 10:1, 9:1, 8:1, 7:1, 6:1, 5:1, 4:1, 3:1, 2:1, 1:1, etc. In certain non-limiting embodiments, the lipid layer may be positively charged and includes hydrogenated soybean phosphatidylcholine and 1,2-dioleoyl-3-trimethylammoniumpropane in a molar ratio of 10:1, 9:1, 8:1, 7:1, 6:1, 5:1, 4:1, 3:1, 2:1, 1:1, etc. Advantageously, a highly charged lipid surface aids the layer-by-layer coating process. To generate a highly charged lipid surface, charged lipids are incorporated into the lipid bilayer in an amount that is sufficient to render the highly charged lipid surface without excessive use of the charged lipids. The molar ratios confer such an advantage, as the charged lipids are in such sufficient amount when used in the presence of a neutral lipid. For example, a negatively charged lipid such as DPPG may be incorporated into a lipid layer containing a neutral lipid of HSPC. The incorporation percentage of DPPG may be 10%, which means that for every 20 mM HSPC used, 2 mM of DPPG is used to render a sufficiently negatively charged surface for the subsequent coating of the positively charged polymeric layer (e.g. chitosan). In such example, the molar ratio of HSPC to DPPG is 10:1. In various embodiments, the lipid layer may be a lipid bilayer.

In various embodiments, each of the more than one positively charged polymeric layers may be or may include poly L-arginine, poly L-lysine, polyallylamine hydrochloride, polyethylenimine, polyamidoamine, or more preferably, chitosan. The chitosan, for example, may have a molecular weight ranging from 15 kDa to 375 kDa, 15 kDa to 310 kDa, 190 kDa to 310 kDa, 15 kDa to 190 kDa, etc. Advantageously, more drugs may be incorporated into the negatively charged drug layer with higher molecular weight polymers used for forming the polymeric layer, as the amount of positively charged groups are higher. The higher number of positively charged groups provide more interaction with a negatively charged drug (e.g. negatively charged insulin) and help confine the drug securely therein. However, if the molecular weight is too high, the particle size may affect cellular uptake, and particle size control may become difficult. Hence, the polymer’s molecular weight, drug loading in drug layer, and the resultant liposome size may have to be considered. A non-limiting example involves chitosan having a molecular weight of 310-190 kDa, which is a medium-sized polymer, and the resultant size of the multilayered liposome having 11 layers of coating remains below 500 nm, which affords desirable cellular uptake as demonstrated in the intestinal epithelial cells (Caco-2) study described in the examples section of the present disclosure.

In various embodiments, the liposome core may include a drug. The drug may be or may include insulin, an insulin-like factor, a growth factor, or a hormonal peptide (e.g. glucagon-like peptide-1 analogue). The growth factor may be or may include adiponectin, FGF21, specific hormonal peptides, etc. Antibodies may also be included as the drug. Such drug may be used for forming the drug layer, where suitable.

In various embodiments, the more than one negatively charged drug layer may have or may constitute a drug loading of at least 1 wt%. For example, the more than one negatively charged drug layer may constitute a drug loading of at least 1.2 wt%. Said differently, all the drug layers or the drug in all the drug layers loaded on the surface of the liposome constitute at least 1 wt%. In various embodiments, the five or more coating layers may include eleven coating layers, and the more than one negatively charged drug layer may have or may constitute a drug loading of at least 10 wt%.

The present multilayered liposome may further include an enteric coating layer formed outer to the outermost coating layer. The enteric coating layer may swell minimally at a pH ranging from 1 to 2, which protects the drug and the polymer layers from acidic environment, such as in stomach.

In various embodiments, the present multilayered liposome may be for use in treating diabetes mellitus.

The present multilayered liposome overcomes the barriers for delivering therapeutic doses of a drug via the oral route, as the drug is protected by the present multilayered liposome during transit through the GI tract and the multilayered liposome is small enough, even after including the drug therein, to initiate cellular uptake and transport across the intestinal epithelial cells. Characteristics of the present multilayered liposome, which is capable of delivering drug and/or proteins in therapeutic amounts, include high loading capacity for the drug and/or protein (e.g. insulin), protection of the drug and/or protein from the GI environment, and enhancement of cellular uptake and transport by the intestinal epithelial cells.

Given the prevalence of patients suffering from diabetes mellitus, oral insulin offers the advantage of non-invasiveness and direct delivery to the liver where glucose homeostasis takes place, reducing the risk of hypoglycaemia and hyperinsulinemia. Carriers such as liposomes have a history of being used in drug delivery, owning to their biocompatibility as well as capacity to accommodate both hydrophilic and hydrophobic drugs. Early findings showed that insulin delivery by encapsulation in a liposome suffered from low solubility of the protein, which adversely affected its entrapment and as a result most of the drug was electrostatically associated with the surface. Drug at the surface of the liposomes are easily compromised as the drug is exposed to elements, different pH environments, biofluids, etc., as the drug migrates through the body. To date, there appears no adequate formulation for oral insulin delivery that meets the therapeutic levels required for daily administration. The present multilayered liposome improves insulin loading and thereby the bioavailability. The present multilayered liposome involves the use of surface coating of nanoliposomes, taking advantage of the high surface area to volume ratio of nanoparticles (NP). Surface modification of liposomes can in principle, alter the properties of the liposome in terms of its stability, release in GI environment, mucosal adherence, drug loading, all of which contribute to improved bioavailability. The present multilayered nanoliposome may include such advantages and is able to deliver insulin across intestinal epithelial cells.

The present multilayered liposome involves a layer-by-layer coating approach/method, which can enhance drug loading using multiple alternating layers of protein and polymer (e.g. counter-ionic polyelectrolyte), and provide sustained release based on the rate of defoliation of the top layers. In one reported example, siRNA-loaded LbL nanoparticles demonstrated cellular uptake and the internalized particle was capable of endosomal escape, resulting in 60% SPARC-gene knock down in FibroGRO cells. In another example, a single layer of siRNA on the nanoparticle surface was able to load 3500 siRNA molecules and co-delivery of the siRNA with doxorubicin-loaded liposome enhanced the serum half-life up to 28 hours and efficacy by 4 fold in vitro. However, the present multilayered liposome, which constitutes a layer-by-layer drug delivery system, allows for an even higher drug loading, especially of macromolecular drugs, and protects the drug and/or bioactives, including siRNA, in the GI environment.

Chitosan is presently used as one of the non-limiting examples for forming the present multilayered liposome, e.g. for oral drug delivery, because of its cationic nature which can increase the mucoadhesiveness and residence time of nanocarrier for enhanced endocytic uptake in the GI tract. The polymer itself also acts as a permeation enhancer, which is able to interact with tight junctions for enhanced permeation of orally administered drugs. The use of chitosan in the present LbL system, as a non-limiting example, not only increases tissue residence time and promotes cellular uptake, furthermore, slow defoliation of the polymer layers can also promote paracellular diffusion of the drug (e.g. insulin) released from the underlying layers, due to the ability of chitosan to transiently open tight junctions. The LbL loading not only helps to preserve the protein structure by lowering the exposure to extreme conditions, but also enables high protein loading while retaining nano dimensions of the carrier. This is a considerable advantage in developing an oral delivery formulation for insulin, which is described in various non-limiting embodiments of the present multilayered liposome and its method of production.

Particularly, the present multilayered liposomes improve the loading and transport of insulin across intestinal epithelial cells by coating insulin layer-by-layer with the help of, e.g. cationic chitosan, onto a liposome surface. Human epithelial colorectal adenocarcinoma cells (Caco-2) were used as an example, for the in vitro model, to study the uptake and transport of LbL-coated liposomes because of their ability to establish apical-to-basal polarity. Chitosan was used as a non-limiting example of the alternating cationic layers to hold the insulin (negatively charged at the pH employed, wherein the pH is maintained at around 9.6 using carbonate-bicarbonate buffer), with the outermost layer being cationic (e.g. chitosan). The cationic outermost layer of the nanoparticle may facilitate trans-cellular transport across the Caco-2 cells, while the “permeation-enhancing” effect of chitosan (free or attached to NPs) facilitates the para-cellular transport of free insulin. The uptake of insulin-loaded LbL-coated liposomes can be analysed by confocal microscopy and the amount of transported insulin was quantified by human insulin ELISA. Bioactivity of the transported insulin can be investigated by glucose uptake assay in differentiated 3T3-L1 MBX adipocytes. In vivo absorption of insulin with the assistance of LbL-coated liposomes was demonstrated following oral gavage in Wistar rat. To elaborate further, the release of insulin from the inner layers of the present multilayered liposome may depend on the speed of defoliation of the outer layer, and the defoliation (or swelling) may be slower at higher pH, for example, where chitosan is less ionized. Conversely, the release may be higher in SGF, wherein the pH may be 1.2 and in such pH, chitosan for example may be highly ionized and swell to a larger extent. Referring to chitosan as a non-limiting example, it is a long chain unbranched polymer having repeating units that include amino groups with a pKa of 6.5. This means chitosan can be positively charged at a pH of 1.2. The human recombinant insulin, for example, has an isoelectric point (pI) of 7, which becomes positively charged, creating repulsion forces that accelerates the penetration of water and charged ions into the underlying layers. At a SIF of pH 6.8, and a pH of 7.4, which are above the pKa of chitosan’s amino groups, both the chitosan and insulin can be neutrally charged. Defoliation or drug release may be slowed down due to the hydrophobic interaction between chitosan and insulin, which prevents water penetration. Advantageously, such release mechanism protects the drugs from being compromised until the multilayered liposome reaches its target site.

The present disclosure also relates to use of the multilayered liposome described according to various embodiments of the first aspect in the manufacture of a medicament for the treatment of diabetes mellitus. The present disclosure also relates to a method of treating diabetes mellitus. The method includes orally administering the multilayered liposome described according to various embodiments of the first aspect or having the multilayered liposome described according to various embodiments of the first aspect to be orally administered. Embodiments and advantages described for the present multilayered liposome of the first aspect can be analogously valid for the present use and method of treating diabetes mellitus mentioned herein, and vice versa. As the various embodiments and advantages have already been described above and examples demonstrated herein, they shall not be iterated for brevity.

The present disclosure further provides for a method of producing the multilayered liposome described according to various embodiments of the first aspect. The method includes providing liposomes each having a liposome core defined by a lipid layer, forming one negatively charged drug layer or one positively charged polymeric layer on the liposome core, depositing one positively charged polymeric layer on the formed negatively charged drug layer or one negatively charged drug layer on the formed positively charged polymeric layer, repeating the deposition of one negatively charged drug layer on the positively charged polymeric layer earlier deposited or one positively charged polymeric layer on the negatively charged drug layer earlier deposited so as to have (i) five or more coating layers surrounding the lipid layer, and (ii) the more than one positively charged polymeric layer and the more than one negatively charged drug layer deposited in an alternating manner, wherein one of the more than one positively charged polymeric layer may be formed as an outermost coating layer, and wherein each of the more than one negatively charged drug layer may include insulin, an insulin-like factor, a growth factor, or a hormonal peptide.

Embodiments and advantages described for the present multilayered liposome of the first aspect can be analogously valid for the present method of producing the present multilayered liposome subsequently described herein, and vice versa. As the various embodiments and advantages have already been described above and examples demonstrated herein, they shall not be iterated for brevity.

In various embodiments, providing the liposomes may include forming a thin film from a solution comprising one or more lipids. This forms the liposome cores. In various embodiments, providing the liposomes may include contacting the thin film with one or more drug solutions in a stepwise manner, wherein the contacting of the thin film with each drug solution is carried out after a time interval from another. This advantageously encapsulates a drug in the liposome core. The drug may be or may include insulin, an insulin-like factor, a growth factor, or a hormonal peptide. The time interval may range from 1 min to 10 mins, 5 mins to 10 mins, 1 min to 5 mins, etc. For example, the thin film may be contacted with each drug solution for 5 mins before contacting with another drug solution.

In various embodiments, the liposomes may be diluted in a carbonate-bicarbonate buffer prior to forming one negatively charged drug layer or one positively charged polymeric layer on the liposome core. Liposomes, e.g. formed using 20 mM HSPC and 2 mM DPPG, may be diluted 20 times for the coating of insulin on the liposome core. For example, 500 µL of HSPC-DPPG liposomes (after using chitosan for coating) may be re-suspended in acidic water at a volume of less than 200 µL, which may be injected into 10 mL of insulin solution to form the insulin coating. Such example provides the 20 times dilution of the original HSPC-DPPG liposomes from 500 µL to 10 mL.

In certain non-limiting embodiments, forming the one negatively charged drug layer on the liposome core may include mixing a carbonate-bicarbonate buffer that includes a drug with the liposomes to form a first mixture, and centrifuging the mixture to obtain liposomes having the negatively charged drug layer formed thereon. In the resultant liposome, the drug (e.g. insulin) for forming the drug layer may have a negative charge.

In certain non-limiting embodiments, depositing one positively charged polymeric layer on the formed negatively charged drug layer may include mixing an organic acid that includes a polymer with the liposomes having the negatively charged drug layer formed thereon to form a second mixture, and centrifuging the mixture to obtain liposomes having the positively charged polymeric layer deposited thereon.

In certain non-limiting embodiments, forming one positively charged polymeric layer on the liposome core may include mixing an organic acid that includes a polymer with the liposomes to form a first mixture, and centrifuging the mixture to obtain liposomes having the positively charged polymeric layer formed thereon.

In certain non-limiting embodiments, depositing one negatively charged drug layer on the formed positively charged polymeric layer may include mixing a carbonate-bicarbonate buffer that includes a drug with the liposomes having the positively charged polymeric layer formed thereon to form a second mixture, and centrifuging the mixture to obtain liposomes having the negatively charged drug layer deposited thereon.

The present disclosure further relates to a multilayered liposome that includes a liposome core defined by a lipid layer, five or more coating layers surrounding the lipid layer, an outermost coating layer which is positively charged, wherein the five or more coating layers may include more than one negatively charged polymeric layer and more than one positively charged drug layer, wherein the more than one negatively charged polymeric layer and the more than one positively charged drug layer may be deposited in an alternating manner, and wherein each of the more than one positively charged drug layer may include insulin, an insulin-like factor, a growth factor, or a hormonal peptide. In the first aspect and its various embodiments, the polymeric layer is positively charged and the drug layer is negatively charged. However, in this subsequent aspect and its various embodiments, the polymer for forming the polymeric layer is negatively charged and the drug for forming the drug layer is positively charged. The multilayered liposome of the first aspect and this subsequent aspect are advantageous in that the charge of the polymeric layer and the drug layer may be versatile. That is to say, the charge of the polymeric layer and the charge of the drug layer may be configured according to various needs. For example, if the pH of the solvent used to dissolve the drug is acidic, the drug may become positively charged and accordingly a negatively charged polymeric (polyelectrolyte) layer may then be used. In other words, the charge of the drug used in the present multilayered liposome for forming the drug layer may be either negative or positive depending on its environmental pH, e.g. pH of the solvent used to dissolve the drug. Hence, the multilayered liposome and their methods of production described in various aspects of the present disclosure advantageously accomodate for drugs that are either negatively or positively charged for forming the drug coating layer.

Understandably, a drug layer can be identified to be negatively or positively charged and have the polymeric layer configured accordingly to form the multilayered liposome of the various aspects described herein. As such, embodiments and advantages described for the multilayered liposome of the first aspect may be analogously valid, where applicable or suitable, for the multilayered liposome of this subsequent aspect described herein, and vice versa. As the various embodiments and advantages have already been described above and in the examples demonstrated herein, they shall not be iterated for brevity.

In various embodiments, each of the more than one negatively charged polymeric layers may include a polymer having a —COOH functional group or a —COO⁻functional group. The polymer having the —COOH functional group or the —COO⁻functional group may include hyaluronic acid, sodium alginate, or a copolymer derived from methacrylic acid, methyl acrylate and/or methyl methacrylate.

In various embodiments, the outermost coating layer may include or may be a positively charged polymeric layer. The positively charged polymeric layer may include or may be chitosan, poly L-arginine, poly L-lysine, polyallylamine hydrochloride, polyethylenimine, or polyamidoamine. Advantageously, the positively charged outermost coating layer may enhance interaction with negatively charged mucin and cell surface, which increases the retention time and absorption of the present multilayered nanoliposome.

In various embodiments, there may be further included an enteric coating layer formed outer to the outermost coating layer, wherein the enteric coating layer swells minimally at a pH ranging from 1 to 2.

The present disclosure further relates to a method of producing the multilayered liposome described according to various embodiments of the subsequent aspect. The method may include providing liposomes each having a liposome core defined by a lipid layer, forming one positively charged drug layer or one negatively charged polymeric layer on the liposome core, depositing one negatively charged polymeric layer on the formed positively charged drug layer or one positively charged drug layer on the formed negatively charged polymeric layer, repeating the deposition of one positively charged drug layer on the negatively charged polymeric layer earlier deposited or one negatively charged polymeric layer on the positively charged drug layer earlier deposited so as to have (i) five or more coating layers surrounding the lipid layer, and (ii) the more than one negatively charged polymeric layer and the more than one positively charged drug layer deposited in an alternating manner, forming an outermost coating layer which is positively charged, and wherein each of the more than one positively charged drug layer includes insulin, an insulin-like factor, a growth factor, or a hormonal peptide. Understandably, embodiments and advantages for the various aspects described above may be analogously valid, where applicable or suitable, for the method of this subsequent aspect described herein, and vice versa. As the various embodiments and advantages have already been described above and in the examples demonstrated herein, they shall not be iterated for brevity.

The word “substantially” does not exclude “completely” e.g. a composition which is “substantially free” from Y may be completely free from Y. Where necessary, the word “substantially” may be omitted from the definition of the present disclosure.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance.

As used herein, the term “and/or” includes any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

### EXAMPLES

The present disclosure relates to a multilayered liposome for a drug to be orally administered. The present liposome circumvents barriers for oral insulin delivery, wherein a considerable factor lies in having an efficient carrier that can protect and enhance the absorption of the protein for achieving therapeutic levels of bioavailability. According to various non-limiting embodiments of the present disclosure, a multilayered polyelectrolyte coating strategy on anionic nanoliposome surface that was able to protect loaded insulin from the harsh gastrointestinal (GI) environment and promote absorption of insulin by the small intestine is developed. High insulin loading (10.7% by weight of liposomal particles) was achieved with alternating layers of chitosan and insulin coated on the liposome surface. The layer-by-layer (LbL) coated nanoliposomes were taken up by Caco-2 cells and intracellular imaging revealed that the internalized nanoparticles were intracellularly trafficked towards the basolateral side of the Caco-2 monolayer. Transport of insulin across Caco-2 cells was enhanced 3-fold with the LbL-coated nanoliposome (over uncoated liposome). Furthermore, the transported insulin triggered glucose uptake in 3T3 L1-MBX adipocytes, thereby demonstrating retention of insulin bioactivity. In rat studies, oral administration of the formulation resulted in peak plasma insulin levels 0.5 hour post oral gavaging. The present disclosure thus provides a foundation to achieve therapeutic levels of insulin in blood with an oral capsule and serves as a promising platform for potential oral insulin delivery. In the present disclosure, the term “particles” may be used interchangeably with “liposomes”, and “nanoparticles” may be used interchangeably with “nanoliposomes”.

The present multilayered liposome, method of producing the multilayered liposome, and uses of the present multilayered liposome, are described in further details, by way of non-limiting examples, as set forth below.

### Example 1A: Materials

Hydrogenated soybean phosphatidylcholine (HSPC), and 1,2-dipalmitoyl-sn-glycero-3-phosphoglycerol, sodium salt (DPPG) were purchased from Coatsome. Chitosan of molecular weight 15 kDa (chitosan 15), 190-50 kDa (chitosan grade 190-50), and 310-190 kDa (chitosan 310-190) were obtained from Sigma-Aldrich. Human recombinant insulin, Triton X-100, coumarin-6, carbonate-bicarbonate buffer, Hanks’ balanced salt solution (HBSS) buffer, 4-(2-hydroxyethyl)-1-piperazineethanesulfonic acid (HEPES) buffer, NaHCO₃, and 12-well Transwell inserts were purchased from Sigma-Aldrich. ELISA kits were purchased from Mercodia. Caco-2 and 3T3 L1-MBX cells were purchased from ATCC. Alexa Fluor (AF) 647 was purchased from ThermoFisher. Glucose uptake-Glo^{Tm} assay was purchased from Promega.

### Example 1B: Liposome Fabrication and Insulin Loading

100 nm liposomes were synthesized following a thin film rehydration method. Briefly, a known amount of HSPC lipids were weighed and dissolved in chloroform and methanol solvents in a 2 : 1 ratio in a round bottom flask. Fluorescent lipid coumarin-6 was added at a 0.5 mol% for the transport study of empty liposomes. Anionic lipid DPPG was added into the organic solvent mixture at 10 mol% for the preparation of a negatively charged surface. Solvents were maintained at constant temperature at 60° C. and allowed to evaporate under a stepwise reduction in pressure and finally stabilized at 20 mbar for an hour in a rotary evaporator (BUCHI Rotavapor® R-100). Insulin solution (10 mg mL⁻¹) containing sodium phosphate dibasic was adjusted to pH 4 using 1 M HCl. A gradient dilution intended to improve the loading was achieved by rehydrating the thin film following a gradient dilution. Generally, insulin solution of 500 µL of 10 mg mL⁻¹, 5 mg mL⁻¹, 2.5 mg mL⁻¹, and 1 mL of 1.25 mg mL⁻¹ was first added to the thin film in a step wise manner with a 5 mins interval in between each addition. Subsequently, 2.5 mL of carbonate-bicarnonate buffer (CBB) (pH 9.6) was added to dilute the liposome solution so that the lipid concentration of the final solution remains at 20 mM.

### Example 1C: Quantification of Encapsulation and Loading Efficiency

Fabricated 100 nm liposomes were ultracentrifuged (ThermoFisher, SORVALL WX ultra) at 50,000 rpm, 4° C. for 30 mins. The concentration of unencapsulated insulin in the supernatant C_{rhINS},_{SN} (mg mL⁻¹), was determined using microBCA assay against a standard curve obtained by serial dilution of insulin of known concentration. The ratio of insulin concentration in the supernatant C_{rhINS},_{SN} (mg mL⁻¹) to the total insulin concentration C_{rhINS},_{TOT} (mg mL⁻¹) can be used to calculate the percentage encapsulation efficiency (EE%) using equation (1):

$$\begin{matrix}\text{EE} \left(\%\right) = \\ \left(1 - \frac{\text{insulin concentration in supernatant}}{\text{original insulin concentration in hydration solution}}\right) \times 100 \left(\%\right)\end{matrix} \qquad \text{Equation (1)}$$

Following the ultracentrifugation step, the pellets were separated from the supernatant and re-suspended in deionized (DI) water. Complete resuspension was achieved by vortex mixing. Thereafter, the liposomal suspension was transferred to a pre-weighed microtube which was then frozen overnight in a -80° C. freezer before being placed in a manifold freeze-dryer for 24 to 48 hours. The mass of the insulin-loaded liposomes was determined from the difference between the mass of microtubes containing powdered liposomes and that of empty microtubes. Percentage loading efficiency (LE%) was calculated using equation (2):

$$\text{LE} \left(\%\right) = \left(\frac{\text{Mass of encapsulated insulin}}{\text{Mass of insulin-loaded liposomes}}\right) \times 100 \left(\%\right) \qquad \text{Equation (2)}$$

Liposome pellet was completely lysed by 1% triton X-100 and insulin concentration both inside the core and in the supernatant were measured by microBCA for calculating the percentage of drug encapsulation. Loading of insulin inside the liposome core was measured by comparing the weight of the encapsulated insulin in the core against the total particle weight and was found to be 0.8 wt%. Overall insulin loading is measured by the weight ratio of the total insulin (inside the core and on the surface) to the total carrier weight, and it is potentially 11.13% by weight. In various non-limiting instances, the amount of drug (e.g. insulin) encapsulated in the core may be 0.6 wt% to 1 wt%. In various non-limiting instances, the total amount of drug in the present multilayered liposome may be more than 0.6 wt%, e.g. 0.6 wt% to 50 wt%, etc.

To calculate the loading of insulin in the layers on the surface of the liposome, insulin was fluorescently tagged with AF647 (Thermo Fisher Scientific) following the manufacturer’s protocol before being coated as the layers on the liposome surface. Standards were prepared by serially diluting known concentrations of the AF647 tagged insulin. The AF647 fluorescence intensity of the final coated particle was compared to that of the particle before coating using a fluorescent microplate reader (Tecan Infinite 200). Coumain-6 serves as an internal standard for normalization of the liposome to ensure equal amounts were used for comparison. Excitation and emission were set at 650 nm and 680 nm for AF647, while they were 480 nm and 530 nm for coumain-6. The mass of insulin loaded as layers on the surface of the liposome was calculated based on the difference in fluorescence before and after coating using fluorescence intensity of serial dilutions of known concentrations of insulin. The mass of the total insulin loaded LbL coated liposome was measured by re-suspending the ultracentrifuged LbL coated liposome in DI and freeze-dried in a pre-weighed microtube. The loading of insulin using the LbL coated liposome was calculated based on equation (3) below:

$$\text{LE} \left(\%\right) = \left(\frac{\text{Mass of insulin loaded as layers}}{\text{Mass of LbL-coated liposomes}}\right) \times 100 \left(\%\right) \qquad \text{Equation (3)}$$

### Example 1D: LbL Coating of Liposomes

Anionic HSPC liposomes containing 10 mol% DPPG lipids were coated with alternating layers of chitosan and insulin based on electrostatic interaction. Odd layers were positively charged chitosan of three molecular weights namely, chitosan 15, chitosan 190-50, and chitosan 310-190. Even layers were negatively charged insulin prepared in CBB buffer. Briefly, odd layers were coated by mixing 0.1% (w/v) chitosan solution in acetic acid (0.1% v/v) with 1 mM liposomes, followed by ultracentrifugation at 50,000 rpm, 4° C. for 60 mins to pellet down the coated particles. Subsequently, the pellets were re-suspended in acidic water (pH 1-2) before injecting into 1 mg ml⁻¹ insulin solution in CBB buffer for the even layers. The process was repeated until 11 layers of coating were achieved for all three molecular weights of chitosan.

### Example 1E: Dynamic Light Scattering Characterization

The liposome suspension was diluted 100 times with deionized(DI) water for analysing its size and zeta potential using a Zetasizer Nano1 (Malvern Instruments, Malvern, UK). Disposable polystyrene cuvettes were used for measuring size, while a folded capillary cell was used for measuring the charge of liposomes.

### Example 1F: Release Study

The release study was performed on LbL coated liposomes in simulated gastric fluid (SGF) pH 1.2, simulated intestinal fluid (SIF) pH 6.8, and phosphate-buffered saline (PBS) pH 7.4 at 37° C. under constant stirring. LbL coated liposomes were placed in a dialysis bag with pore size 300 kDa. Release samples were collected and quantified by ELISA.

### Example 1G: Cellular Uptake

Caco-2 cellular uptake was studied by flow cytometry and confocal microscopy. Caco-2 cells were seeded in 6-well plate with glass cover slip, maintained for at least 21 days before treating with coumarin-6 tagged LbL liposomes. After 4 hours of treatment, cells grown on the coverslip in the 6-well plate were washed twice with PBS, fixed with ice cold methanol and perforated with 0.1% Triton-X, before staining immunostaining with anti-claudin-1 antibodies. After staining, coverslips were inverted and mounted using VectaShield mounting media onto a microscopy slide for imaging by ZEISS confocal laser scanning microscope LSM 710.

### Example 1H: Transport Study

To establish an in vitro cell model for the transport study, Caco-2 cells were grown on 12-well Transwell inserts with 0.4 µm pore size with TEER values monitored every second day for the development of a monolayer for 21 days. Caco-2 cells were cultured in T75 flasks with Dulbecco’s Modified Eagle’s Medium (DMEM) high glucose medium containing 20% fetal bovine serum (FBS), 1% penicillin streptomycin solution (Pen strep), and 1% essential amino acids and harvested when the cells were 70-80% confluent. Cells were seeded at 300,000 cells per well on a 12 mm permeable membrane support. Medium was changed every other day post seeding and transepithelial electrical resistance (TEER) values were recorded using epithelial voltohmmeters (EVOM) equipped with “chopstick” electrodes (World Precision Instruments, Sarasota, FL). The transport experiment was performed 21 days post-seeding when the TEER values reached above 300 Ω cm². HBSS was prepared by dissolving 9.7 g of HBSS, 4.7 g of HEPES, and 0.35 g of NaHCO₃ in 1 litre of DI water. 1 M NaOH was used to adjust the pH of the solution to pH 7.4 and the buffer solution was sterile filtered using a 0.22 µm pore size membrane. The transport experiment was carried out following an established protocol. Briefly, 500 µL of 1 mM LbL coated liposomes were added in the apical compartment and 1.5 mL of the buffer were added into the basal compartment of the 12 well insert. Sampling was performed at 0, 1st, 2nd, 3rd, and 4th hour with complete buffer replacement from the basal compartment. Alamar Blue (Biorad) assay was performed immediately after the transport study on Caco-2 cells cultured on a Transwell membrane. Briefly, Caco-2 cells after treatment were incubated with 50 µl of Alamar blue dye diluted with PBS at 37° C. for 6 hours. The results were obtained using a microplate reader using an excitation wavelength of 560 nm and emission wavelength of 590 nm.

### Example 11: Glucose Uptake Study

3T3 L1-MBX fibroblasts were differentiated into adipocytes following the recommended protocol from the Glucose Uptake-Glo™ kit. 3T3 L1-MBX fibroblasts were maintained in DMEM containing 10% FBS and 1% antibiotic-antimicotic and used for differentiation within 10 passages. On day 1, cells were seeded at 20 000 cells per 100 µl in a 96-well plate and maintained in maintenance medium (DMEM containing 3% fetal bovine serum and 1% antibiotic-antimicotic) for 4 days. On day 5, medium was replaced with 100 µl of differentiation medium-I which was prepared by adding differentiation drugs including insulin (1 µg mL⁻¹), isobutylxanthine (0.5 mM), dexamethansone (1 µM), and rosiglitazone (2 µM) to the maintenance medium. Medium was replaced every 2 days. On day 12, medium was replaced with 100 µl of differentiation medium-II which was prepared by adding insulin (1 µg mL⁻¹) to the maintenance medium. On day 14, medium was replaced back with maintenance medium and maintained for another 8 to 11 days with medium replacement every 2 days until the glucose uptake experiment was performed. Insulin response was evaluated for glucose uptake assay upon maturation of the 3T3 L1-MBX adipocyte, and luminescence of cells treated with transported insulin and PBS control was measured using a microplate reader (Tecan Infinite 200). Cells were treated with known concentrations of insulin for generating the standard insulin response curve for comparison.

### Example 1J: Quantification of Transported Insulin

The amount of insulin in the collected buffer from the transport study was quantified for transported insulin by ELISA (Mercodia), which was performed according to the manufacturer’s protocol using a known amount of human recombinant insulin (Sigma Aldrich) as the standard.

### Example 1 K: Pharmacokinetic Study in Vivo

The pharmacokinetics of orally administered LbL coated liposome formulation was evaluated in adult nondiabetic male Wistar rats that had undergone 12 hours fasting. For the solution group, lyophilized formulation was reconstituted in DI water and administered at 320 IU kg⁻¹ before oral gavage dosing. Lyophilized formulation and bare insulin powder were loaded in enteric coated size 9 capsules (Torpac) and dosed at 43 IU kg⁻¹. Blood was collected by inserting a tail vein catheter into the lateral tail vein using a BD Insyte 22-gauge needle. After inserting the catheter, the samples were withdrawn from the catheter. To maintain the patency at each sample 100 µL of heparinized saline (10 U mL⁻¹) was flushed. At each time point when the catheter cap was opened and flushed with heparinized saline, the initial 2 drops of blood was discarded and 200 to 300 µL of blood was collected. After sample collection the cap was closed by flushing with 200 µL of heparinized saline. The same procedure was repeated for all time points. Blood was collected in microtubes and kept on ice and centrifuged after collecting all samples. The microtubes were centrifuged at 5000 relative centrifugal force (rcf) for 10 mins at 40° C. to separate the plasma. The plasma was collected into two aliquots of approximately equal volume. The plasma was transferred immediately to -80° C. Permission to operate animal experiments was obtained with Institutional Animal Care and Use Committee (IACUC) Service Protocol number #181313, entitled for evaluation of novel compounds to assess toxicity and efficacy of pharmacokinetic/pharmacodynamic parameters in mouse models. This study was performed in strict accordance with the NIH guidelines for the care and use of laboratory animals (NIH Publication No. 85-23 Rev. 1985) and was approved by the Institutional Animal Care and Use Committee (Singapore).

### Example 2A: Results - Fabrication and Characterization of Insulin-Loaded LbL Coated Liposomes of the Present Disclosure

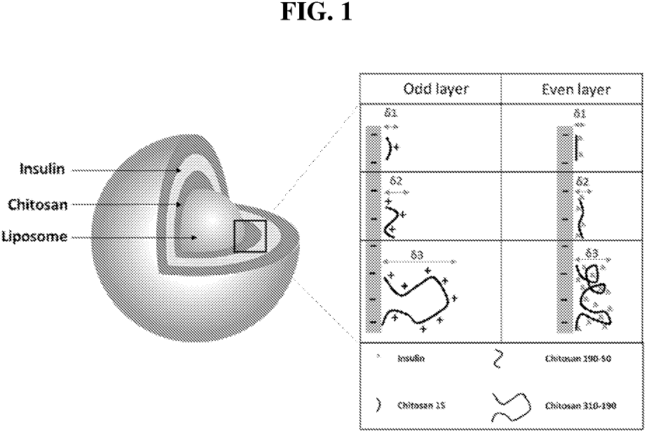

The limited loading of macromolecular drugs by nanoliposomes has been hampering its translation for the delivery of therapeutic proteins such as insulin. Diasome pharmaceuticals’ HDV (Hepatocyte-Directing Vesicle) liposome which may be currently in clinical trials, encapsulates only 1% of insulin with the majority staying in the free form outside the carrier. To improve loading, a layer-by-layer approach was used to modify the nanoliposome surface with multilayers of oppositely charged insulin and chitosan layers, using an anionic liposome core (HSPC/DPPG) (FIG. 1). The liposome aqueous core was loaded with insulin during thin film rehydration before surface modification. Since delaying the release of insulin in the GI environment and promoting cellular uptake were desired for the multilayered nanoparticle, chitosan was coated as the odd layers to serve as the limiting factor for controlling the diffusion of insulin from both the aqueous core and the multilayers. Chitosan of 3 different molecular weights namely chitosan 15 kDa, chitosan 190-50 kDa, and chitosan 310-190 kDa were used to compare the differences in drug loading capacity. Chitosan with the highest loading capacity was selected for subsequent in vitro and in vivo studies.

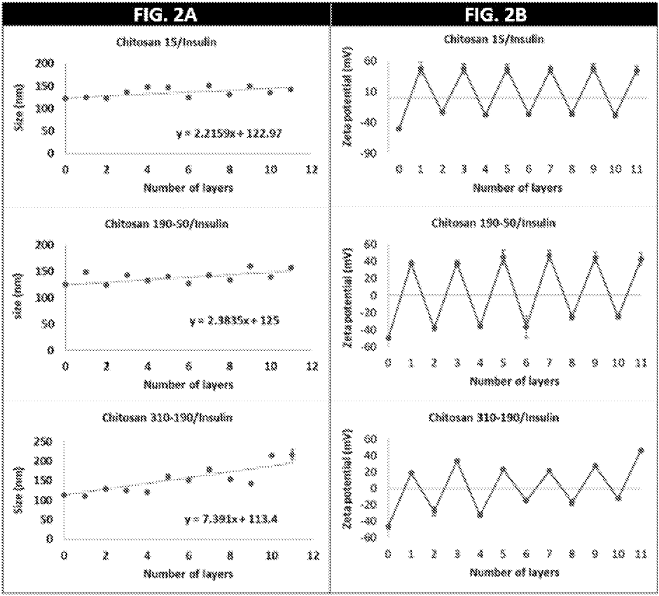

The zeta potential measurement shows charge reversal when the anionic liposome surface was modified with chitosan of all three different molecular weights and the pattern repeats as the coating is continued for the subsequent insulin and chitosan layers (FIG. 2B). This indicates that the coated liposomes were stabilized by the repelling force generated by the vesicle surface charge and the coating technique is effective regardless of the number of repeating units of the cationic polymer. Comparing the hydrodynamic radius between the LbL-liposomes coated with the three molar masses of chitosan, increasing hydrodynamic radius was observed as the number of layers increased, and the increment was larger for LbL-liposomes coated with higher molecular weight chitosan. The trend line for each graph in FIG. 2A shows that the average thickness per layer of coating increased from 2.2 nm to 7.4 nm as the polymer molar mass increased from 15 kDa to 310-190 kDa. This is likely due to “excess” cationic charge on the longer chains, which may result in uneven binding to the negatively charged insulin, resulting in coiling and extension into space of the chitosan layer. Interestingly, the hydrodynamic radius of both the insulin layers and chitosan layers showed generally an increasing trend as the number of layers increased; if examined separately, however, the size increment after insulin layer addition is generally smaller than that of the chitosan layers. This could be due to the structural difference between chitosan and insulin. Chitosan is a long chain of unbranched polymer consisting of repeating units of amino groups with pKa 6.5, making the polymer positively charged in weakly acidic conditions. On the other hand, insulin is a protein that has its unique 3-dimensional shape with an overall negative charge when dissolved in a buffer of pH higher than its isoelectric point. The deposition of chitosan layers on the liposome surface results in partial attachment of the long polymer chains with a portion of it extending out into the solution. This creates binding space for the incorporation of insulin, making the protein layer more likely to be condensed because of the molecular interactions such as intra-molecular hydrogen bonding and hydrophobic interaction, and resulting in a smaller size increment for the insulin coating compared to that of chitosan.

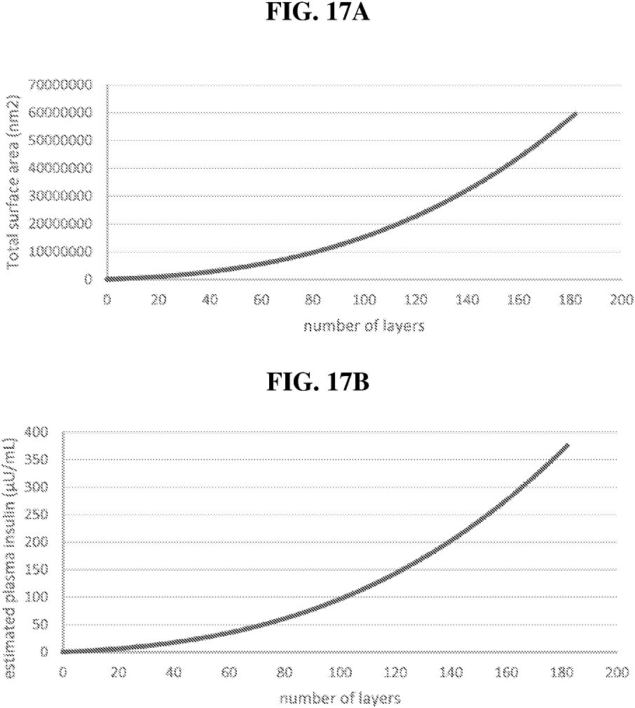

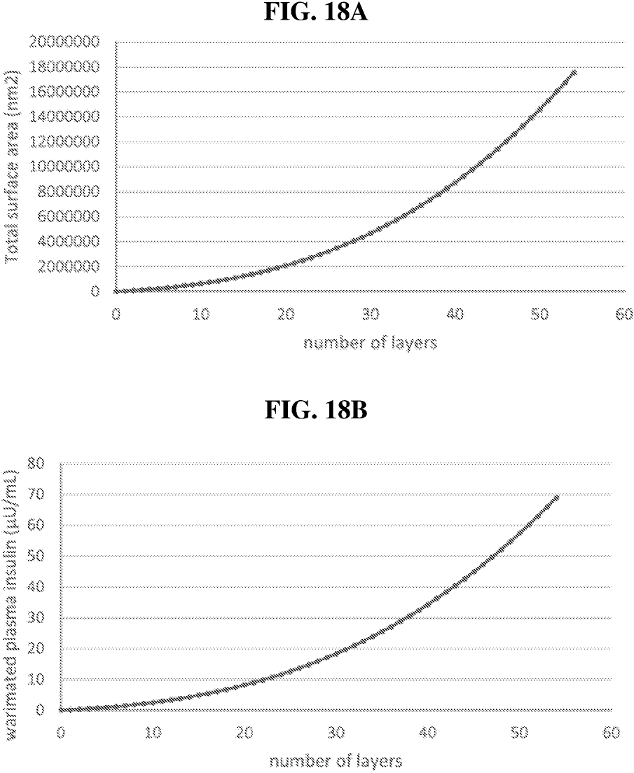

As mentioned above, the average thickness per layer increase in diameter can be estimated from the slopes of the trend lines in FIG. 2A. It ranges from 2.2 to 7.4 nm as the molecular weight of chitosan varies. The liposome core possessing a diameter of 100 nm was fabricated via thin film rehydration followed by high pressure extrusion. If the resultant LbL-coated liposome’s diameter was configured to be 500 nm or less, which may be considered optimal for a desirable drug uptake, then the resultant liposome may be estimated to potentially contain a coating having 54 to 182 alternating layers of chitosan/insulin (about 27 to 91 layers of insulin), depending on the molecular weight of chitosan chosen. The total surface area of the resultant liposome and the estimated concentration of insulin loaded therein using 15 kDa and 310-190 kDa chitosan are plotted in FIGS. 17A to 17B and 18A to 18B, respectively. Calculations of the loading capacity of insulin for liposomes using chitosan of different molecular weights are described as follows.

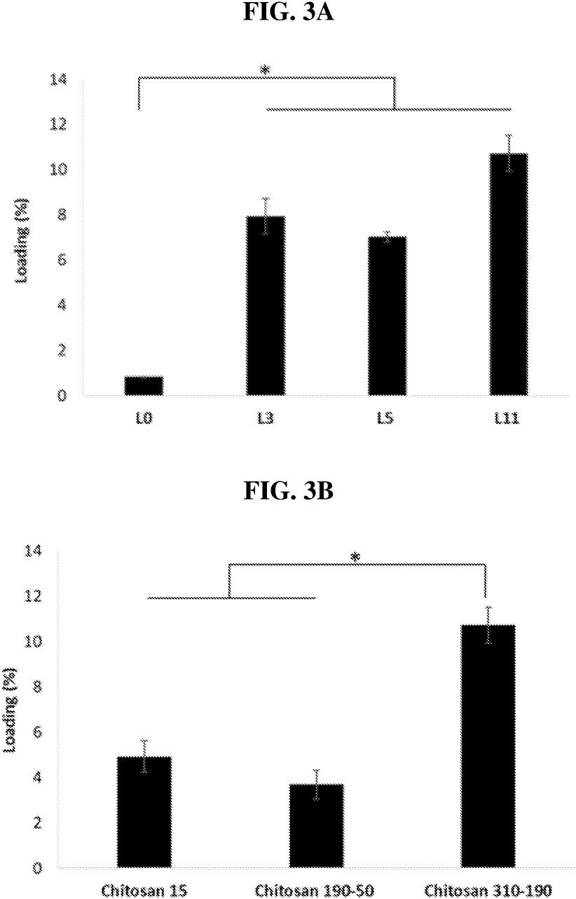

For chitosan molecular weight 15 kDa:
the average thickness per layer increase in diameter is 2.2 nm
The total coating thickness = 500 nm - 100 nm = 400 nm
Total number of layers = 400 nm divided by 2.2 = 182 layers
Number of insulin layers = 182 divided by 2 = 91 layers
Current 11-layer LbL-coated liposome (5 layers insulin) loading capacity = 4.9% (also see FIG. 3B)
A 182-layer LbL-coated liposome (91 layers of insulin) loading capacity = (91 × 4.9)/5% = 89%

For chitosan molecular weight 310-190 kDa:
the average thickness per layer increase in diameter is 7.4 nm
The total coating thickness = 500 nm - 100 nm = 400 nm
Total number of layers = 400 nm divided by 7.4 = 54 layers
Number of insulin layers = 54 divded by 2 = 27 layers
Current 11-layer LbL-coated liposome (5 layers insulin) loading capacity =10.7% (FIG. 3B)
A 54-layer LbL-coated liposome (27 layers of insulin) loading capacity = (27 × 10.7)/5% = 58%

### Example 2B: Results - Loading of Insulin in LbL Coated Liposome

Two groups of the LbL coated liposome were studied for insulin loading using a fluorescence-based method. The first group addresses the effect of increasing the number of coating layers on the loading of LbL-liposomes, whereas the second group addresses the effect of different molecular weights of chitosan on the loading of LbL-liposomes while keeping the number of layers constant. In the first group, loading of insulin for the LbL coated HSPC/DPPG liposomes (L3, L5 and L11) was significantly higher (*p < 0.05) compared to the uncoated HSPC/DPPG liposomes (L0) in FIG. 3A. L11 (11-layers) with 5 layers of insulin coating on the liposome surface, significantly improved the loading by 13x compared to the conventional thin film rehydration method. While comparing the loading of insulin between the LbL-liposomes prepared from different molecular weights of chitosan, the loading of LbL-liposomes prepared from chitosan 310-190 reached 10.7% by weight which was significantly higher (*p < 0.05) compared to LbL-liposomes prepared from chitosan 190-50 and chitosan 15 (FIG. 3B). This is simply due to increases in charge density that enables condensation of more insulin molecules per layer.

### Example 2C: Results - Release and Stability of the LbL Coated Liposome in Simulated Bodily Fluid

During the GI transit, insulin experiences a variation of pH from a highly acidic pH 1.2 in the stomach, to pH 6.8 in the small intestine, to finally a neutral pH 7.4 if the drug overcomes all the barriers and reaches the blood. LbL coated liposomes are targeted at protecting insulin during its transit through the GI tract and facilitating its absorption in the small intestine. In order to study the effectiveness of these particles in protecting insulin and the stability of these particles during the GI transit, in vitro release studies were assessed in simulated gastric fluid (SGF pH 1.2), simulated intestinal fluid (SIF pH 6.8), and Phosphate-buffered saline (PBS pH 7.4).

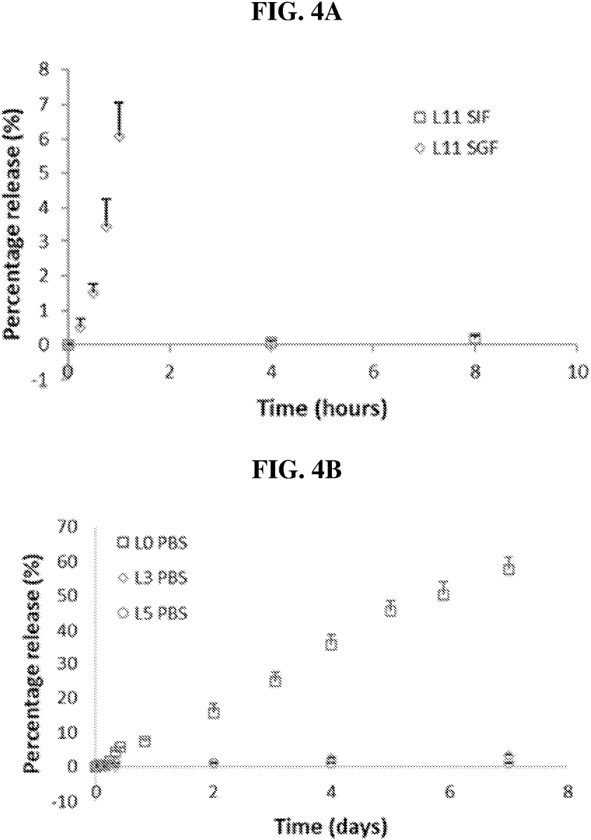

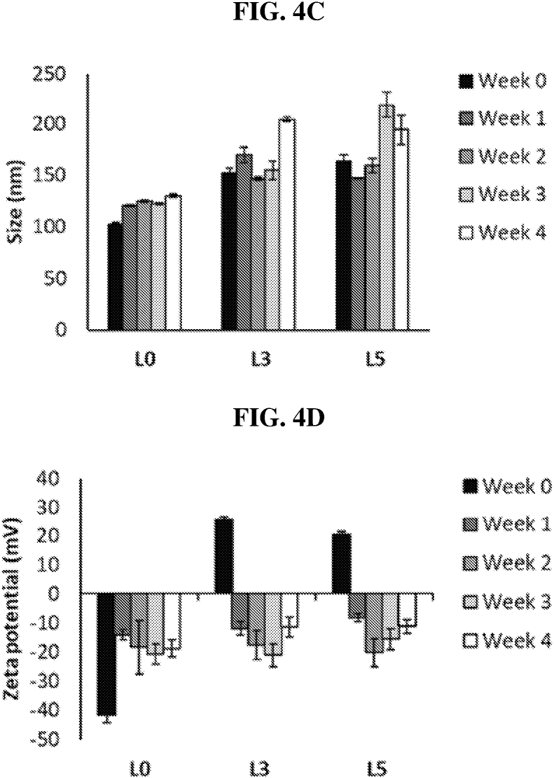

First, a 11-layer LbL-coated nanoparticle with 5 layers of insulin and 6 layers of chitosan was considered. If the 5 layers are distributed equally, each layer is 20% of the total amount of insulin. At pH 1.2 (SGF), about 6% insulin is released in one hour (FIG. 4A), implying that the chitosan has swollen enough to allow for some release of insulin. At the higher pH of 6.8 (SIF), this release is much slower, with less than 0.2% of insulin being released in 8 hours, consistent with lowered swelling of the chitosan (lower degree of ionization) at pH 6.8 compared to pH 1.2 (100% ionization). Turning to the effect of layering (FIG. 4B), it was found that the percentage of insulin released is roughly the same, regardless of it being 3-layers (1 insulin layer) or 5-layers (2 insulin layers). The 3-layered system, however, releases a higher percentage over 7 days. Nevertheless, for 11-layered systems, the release is very slow at pH 6.8 implying that the insulin stays mostly encapsulated while in the small intestine. The release is similar at pH 7.4, observable for a low degree of ionization of the chitosan. Looking at the zeta potential data (FIG. 4D), it appears that the defoliation of the outermost chitosan layer is completed in 7 days. Chitosan is a long chain of unbranched polymer consisting of repeating units of amino groups with pKa 6.5, making the polymer positively charged at pH 1.2. The human recombinant insulin used in this study has an isoelectric point (pI) of 7, which becomes positively charged when the surrounding pH is lower than its pI. At pH 1.2, defoliation or drug release is faster because both chitosan and insulin are positively charged, creating repulsion force which accelerates the penetration of water and charged ions into the underlying layers. At SIF pH 6.8 and pH 7.4 which is above the pKa of chitosan’s amino groups, both chitosan and insulin should be neutrally charged. Defoliation or drug release is slowed down due to the hydrophobic interaction between chitosan and insulin which prevents water penetration.

### Example 2D: Results - Caco-2 Uptake and Transport of LbL Coated Liposome

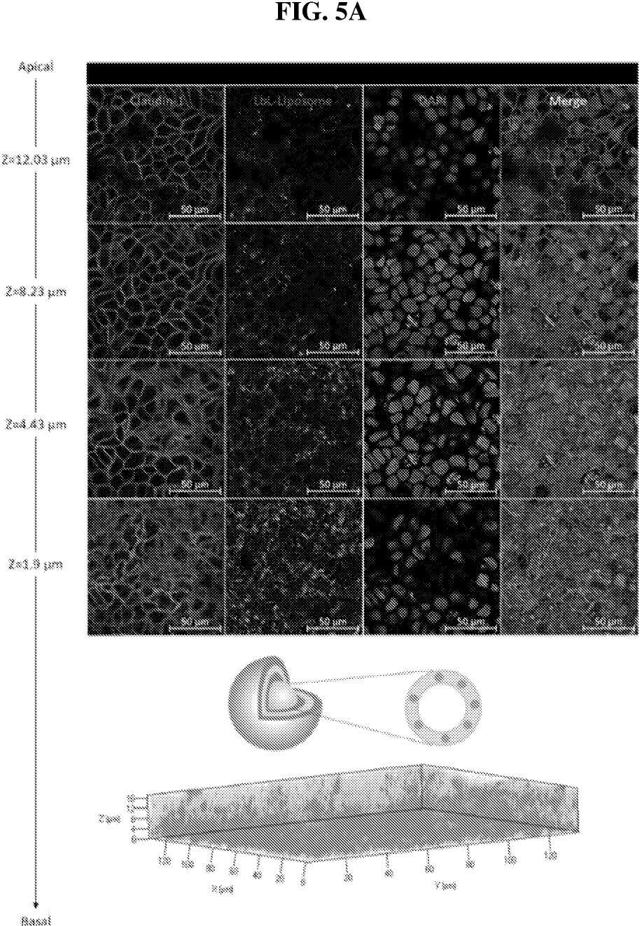

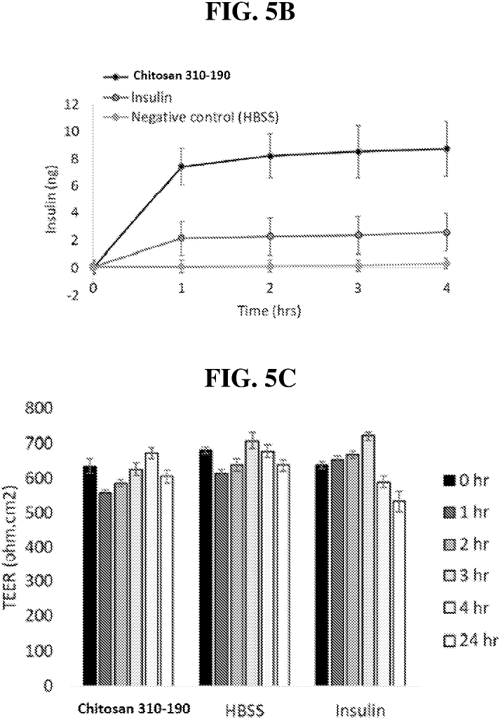

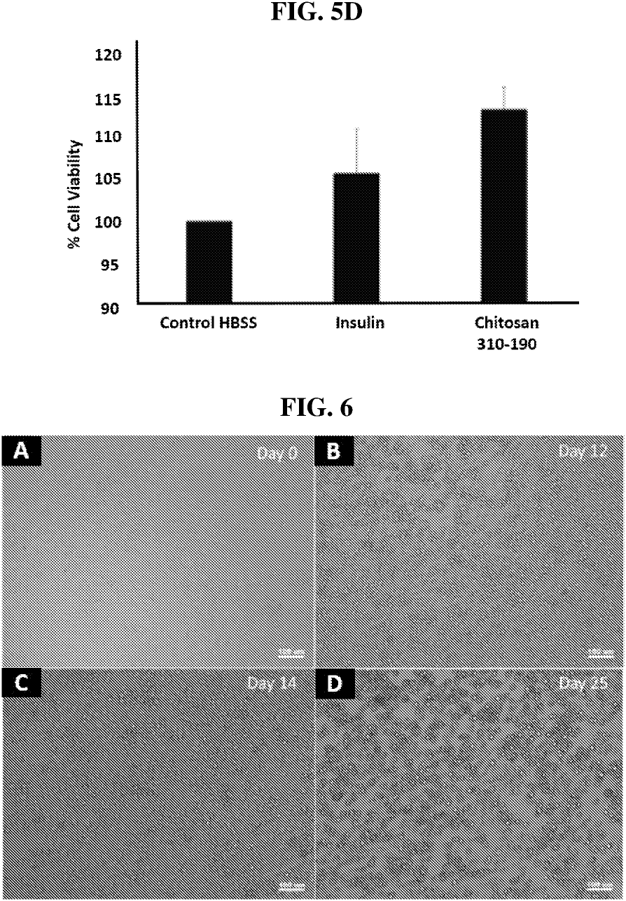

In order to determine the biological relevance of the fabricated insulin-chitosan liposomes, the LbL coated liposome formulation was tested on human epithelial colorectal adenocarcinoma cells in culture (Caco-2 cells) to investigate the ability to be taken up and transported across the intestinal epithelial cells in vitro. Caco-2 cells were seeded on a glass coverslip and maintained for 21 days with medium replacement every 2 days before being treated with the LbL coated liposomes. The lipid bilayer of the liposome inner core (11-layered system) was fluorescently tagged with coumarin-6 for monitoring intracellular trafficking (FIG. 5A). Confocal microscopy revealed that these nano-sized LbL coated liposomes were heavily up-taken by Caco-2 cells and the nanoparticles translocated to the basal side of the monolayer after 4 hours of treatment as shown in the 3D rendering in bottom image of FIG. 5A. The Z-stack analysis allowed visualization of the monolayer on different planes (from the apical to the basolateral surface) and thus showed the intracellular position of the endocytosed nanoparticles at different depths. Images at different Z-positions, which represent the distance away from the bottom of the monolayer, reflected that the endocytosed nanoparticles concentrated near the bottom of the monolayer. As the Z-position decreased from 12.03 µm to 1.9 µm, the green fluorescence from the nanoparticles gradually increased (FIG. 5A), indicating that the nanoparticles have been intracellularly trafficked from the apical side to the basal side of the caco-2 cells. The outermost layer of the LbL coated liposome (11-layered) is cationic, which enables cellular uptake due to favourable interaction with the negatively charged cell surface.

Transport of insulin across the Caco-2 cells was studied by ELISA (FIG. 5B). The amount of insulin being transported across the Caco-2 cells with the help of the LbL carrier was more than 3x higher than bare insulin. TEER measurement during the 4 hours of transport indicated that no significant changes to the tight junctions occurred during the uptake and transport of the LbL coated liposomes as the TEER values remained at a value above 500 Ω cm² during and 24 hours after treatment (FIG. 5C). Since the particles are stable in SIF conditions with minimal insulin release, and the tight junction was intact as indicated by the increasing TEER values, this enhanced transport of insulin is likely to be the result of carrier facilitated endocytic uptake of the LbL coated liposome, followed by exocytosis of intracellularly released insulin while crossing the Caco-2 monolayer. Alamar Blue assay was performed immediately after the treatment to investigate cytotoxicity of the LbL nanoparticles. The results showed that the percentage cell viability was higher when cells were treated with insulin alone or LbL nanoparticles compared to cells treated with HBSS control (FIG. 5D). This increase in percentage viability could be due to the growth promoting effect of insulin released from the LbL nanoparticles, further indicating that the LbL formulation was safe for use in vitro.

### Example 2E: Results - Glucose Uptake by 3T3 L1-MBX

3T3 L1-MBX fibroblasts were differentiated into adipocytes for the study of glucose uptake of the transported insulin. 3T3 L1-MBX fibroblasts were maintained in DMEM containing 10% fetal bovine serum and 1% antibiotic-antimicotic and used for differentiation within 10 passages. Cells were maintained in maintenance medium (DMEM containing 3% fetal bovine serum and 1% antibiotic-antimicotic) for 4 days prior to treatment with maintenance medium containing insulin (1 µg mL⁻¹), isobutylxanthine (0.5 mM), dexamethansone (1 µM), and rosiglitazone (2 µM) to the maintenance medium, followed by treatment with maintenance medium containing insulin (1 µg mL⁻¹). Medium was replaced back with maintenance medium after 2 days and maintained for another 8-11 days with medium replacement every 2 days until the glucose uptake experiment was performed. Morphologically, the elongated fibroblast gradually altered into spherical fat storing cells with big intracellular vesicles and reduced size of the cytoplasm (FIGS. 6A to 6D). Oil red staining indicated that the cells had been fully differentiated into adipocytes (FIGS. 6E to 6F). Upon maturation, the fully differentiated 3T3 L1-MBX cells were treated with the basal compartment fluid collected from the transport study. An insulin titration curve was first established by measuring the glucose response of matured adipocytes when treated with known concentrations of insulin. From FIG. 6G, a typical sigmoidal curve was obtained indicating that the cells were responsive to glucose when treated with the insulin used for fabricating LbL coated liposome. In FIG. 6H, the luminescence detected for cells treated with the transport fluid was about 3.5× higher than the cells treated with PBS negative control. The glucose response was correlated to 1 to 100 nM of insulin based on the insulin titration curve, suggesting that a substantial amount of insulin after crossing the intestinal epithelial cells was bioactive in triggering the glucose uptake in adipocytes. The results from the glucose uptake study suggests that LbL coated liposomes were effective at protecting the insulin during its intracellular trafficking by Caco-2 cells, and the transported insulin has retained its bioactivity while crossing the intestinal epithelial Caco-2 monolayer.

### Example 2F: Results - Lyophilization of LbL Coated Liposome

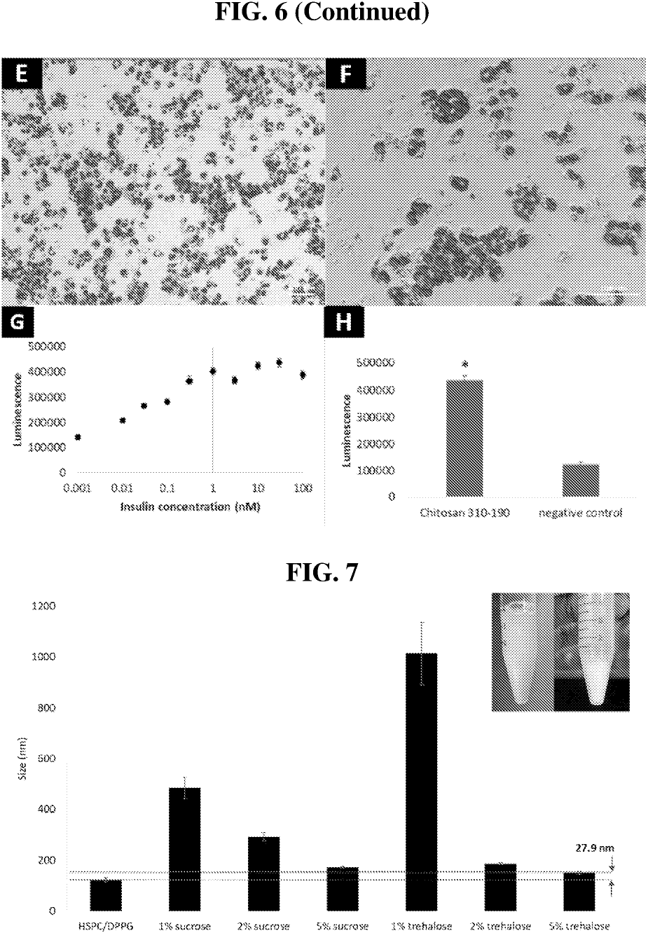

Lyophilisation was carried out to increase the shelf-life of the formulation for extended storage stability. Reconstituted lyophilized formulation was used throughout the animal study. The LbL coated liposome also demonstrated excellent stability during lyophilisation when an appropriate cryo-protectant was added (FIG. 7). Comparing between two common cryo-protectants sucrose and trehalose, increasing the concentration ensures better stability during lyophilisation and size recovery upon reconstitution in water. Trehalose served as a better cryoprotectant compared to sucrose when applied at 5% during lyophilisation since the size recovered closest to the original size with only 27.9 nm increment upon reconstitution. Trehalose has been used as a cryoprotectant in stabilizing nanoparticles during freeze drying due to its advantages such higher glass transition temperature T_{g}, lower hygroscopicity and the ability to form flexible hydrogen bonds with nanoparticles which allows easy removal of these sugars from the nanoparticle surface after lyophilisation.

### Example 2G: Results - Pharmacokinetics of Plasma Insulin Level in Vivo

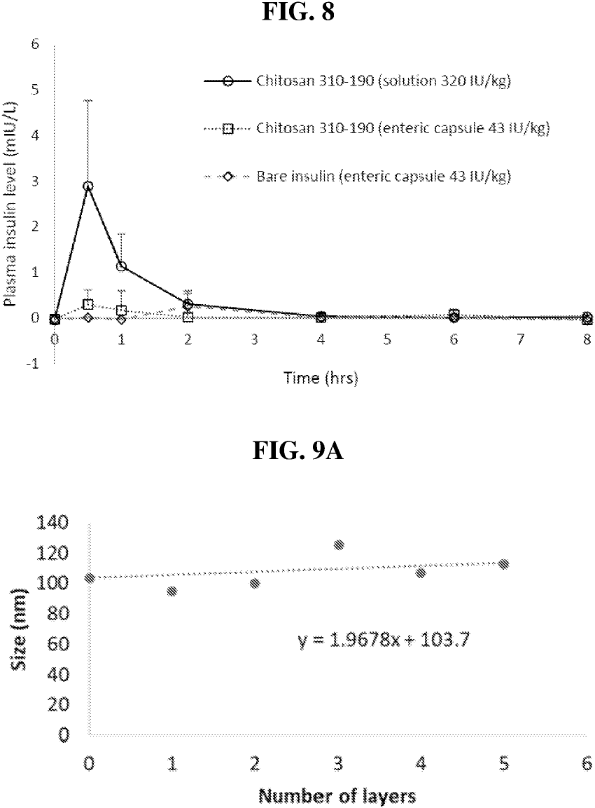

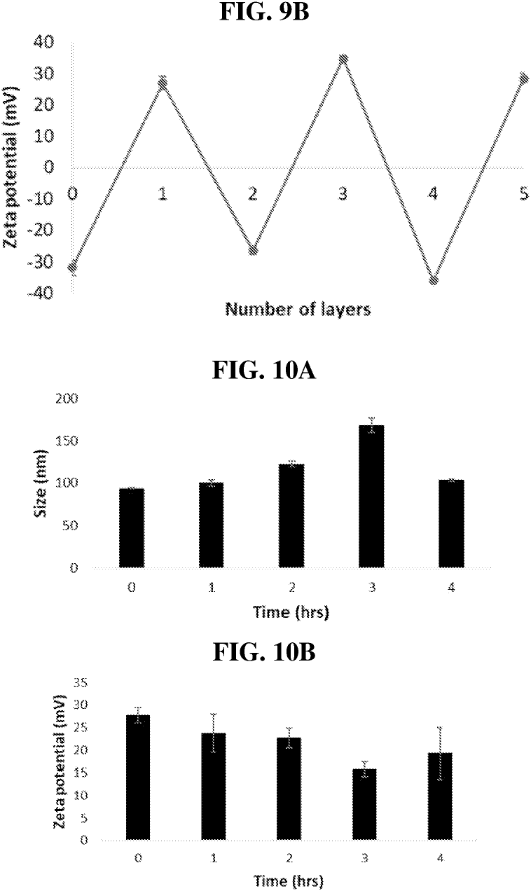

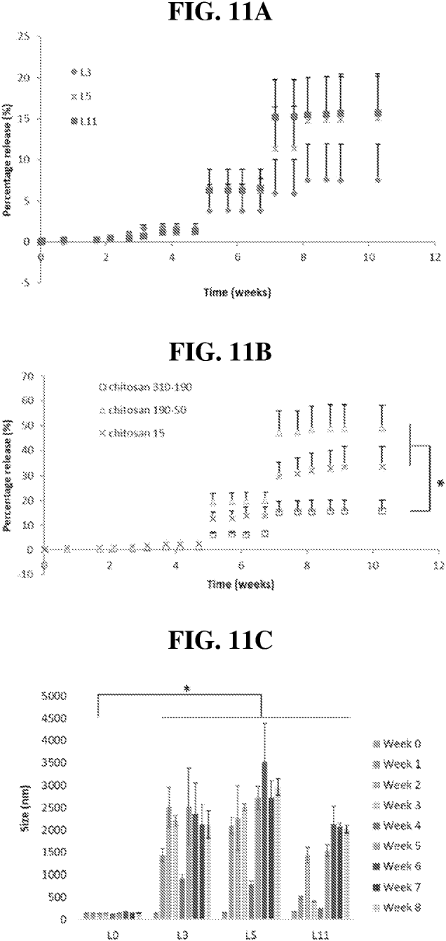

To study the pharmacokinetics of insulin absorption and elimination, a total of 12 Wistar rats with 4 animals per treatment group was used. The LbL coated liposome formulations were fed to over-night fasted rats via oral gavage in solution and capsule form. Human insulin ELISA (Mercodia, Sweden) with no cross-reactivity to endogenous rat insulin (0.7%) was used to measure the blood distribution of human insulin loaded inside the LbL coated liposome. Lyophilized LbL-liposome formulation was applied at maximum dosage in both solution and capsule form to select the group with a positive outcome, based on which a potential method of delivery is be selected for dosage optimization and efficacy test. Oral administration of 320 IU kg⁻¹ insulin loaded chitosan 310-190 LbL nanoparticles (11-layered system) in solution resulted in a rapid increase in plasma insulin concentration which peaked at 0.5 hours with maximum absorption of close to 3 mIU L⁻¹ and a subsequent decrease to the baseline level within the next 3.5 hours due to elimination (FIG. 8). On the other hand, no significant insulin was detected in the plasma when the same formulation was administered in an enteric coated capsule at 43 IU kg⁻¹. One possible reason could be that the formulation loaded inside the capsule was not released during the short passage time in the GI of the rat, causing the formulation to be excreted before being absorbed. It is also possible that the dose given in the capsule was not high enough to deliver measurable levels of insulin in the plasma. Furthermore, the volume and concentration of the solution group might also influence the relationship between applied dosage and plasma insulin level. Considering that in an animal model, absorption of intact orally administered protein drugs is virtually impossible as macromolecules are digested into their simplest units before absorption, any amount of insulin detected in the blood means the LbL nanoparticles has overcame significant amount of hurdles in delivering insulin across intestinal epithelial cells.

### Example 3: Discussion of the Results

In the examples of the present disclosure, a facile layer-by-layer method for loading large amounts of insulin on the surface of a nanoliposome is described. The LbL nanoparticles outperform the conventional liposome in terms of (1) drug loading, (2) protection against GI environment, and (3) penetration of intestinal epithelium with retention of bioactivity. The release of insulin from the inner layers in this LbL system is dictated by the speed of defoliation of the outer layer, and this defoliation (or swelling) is slower at higher pH, where the chitosan is less ionized. Conversely, the release is expected to be higher in the SGF, with a pH of 1.2, where the chitosan is highly ionized and therefore swells to a larger extent. Thus, it is expected that about 6% of the insulin in the nanoparticle may be lost in the stomach, but 94% should be still encapsulated as it passes into the small intestine. This loss may be decreased by using a capsule with enteric coating that has to be precisely controlled for releasing the content at the right section of the small intestine.

Most of the existing nanomedicines suffer from low drug loading with almost all formulations, having the drug content being less than 10% of the NP weight. This has been a stumbling block in translation, 8 because even if sufficient absorption of NPs occurs in the small intestine, drug bioavailability is still inadequate for therapeutic effect. For example, Diasome pharmaceuticals’ HDV liposome can only hold 1 IU of insulin per 1 mg of HDV, resulting in a formulation consisting of 99% free insulin and only 1% is associated with the carrier. Trying to load insulin into the core of the nanoliposome has not met with any success because of (1) the large size of the protein, (2) difficulty of driving insulin into the core, and (3) limited aqueous core capacity of the liposome. Due to the abovementioned reasons, protein tends to sit on the surface of the liposome instead of moving inside the core during thin film rehydration. Other methods such as active loading have been extensively explored, but due to the size, charge, and hydrophilic nature of the protein, little success has been achieved as diffusion across the lipid bilayer after carrier formation was almost impossible.

In the present technology, coating only 5 layers of insulin on the liposome surface using Chitosan 310-190 kDa results in a 10.7% loading by weight which is considered relatively high for a nanocarrier. Increasing the number of coating layers results in increasing amount of loaded insulin per particle. This implies that even if insulin release rates remain the same, greater cumulative amounts of insulin are in fact released with the higher loading. Furthermore, since insulin is loaded on the surface of a spherical liposome, loading of insulin may be size and surface area dependent. The surface area of a spherical nanoparticle is 4πr², which is directly related to insulin loading and have an exponential relationship with the particle radius. Loading increases as the size of the particle increases, which provide a larger surface area to accommodate more insulin as the number of layers increase. In addition, most current nanocarriers only load insulin inside the core, which has a limited capacity and drug loading becomes extremely challenging when the carrier size is down to the nanometer. In contrast, the biggest advantage of the present technology is that a new approach supporting loading of protein on the nanocarrier surface was developed, the successful application of which allows enormous room for improving loading by extending the protein layer into the vast outer aqueous space, significantly changing the way a protein drug can be loaded. The timeframe of release of the LbL coated nanoliposome is superior in SGF, SIF, and PBS due to the direct complexation of insulin which stabilizes the protein within the layers via electrostatic interactions. The stability of the LbL coated liposome was excellent in PBS pH 7.4 with only 50-60 nm increase in size over a period of 4 weeks at 37° C. (FIGS. 4C and 4D). This was not achieved in the well-established chitosan tripolyphosphate (TPP) nanoparticles as the particles were unstable at pH 7 and above with almost 60% release at pH 7.4 within a few hours. As a result, the compromised particle also loses its ability to be taken up by cells.

The LbL coated nanoliposome has a cationic outermost layer that facilitates its association, uptake and transport by the intestinal epithelial cells, specifically Caco-2 cells. In the present study, chitosan was selected as a coating layer to improve insulin loading because of its ability to form ionic complexes with negatively-charged drugs, and its biodegradability as well as reported biocompatibility. In a couple of studies, free chitosan has been reported to act as a permeation enhancer, enabling increased transport via the paracellular pathway: enhanced enteric absorption of insulin and a hypoglycemic effect was observed after oral administration in mice and rats. It was postulated that chitosan enhances paracellular transport of insulin by mediating with the tight junction protein claudin-4, thereby opening up the junctional space for the passage of insulin. However, the present studies do not demonstrate an enhancement of paracellular transport by chitosan-coated nanoliposomes. TEER measurement during the in vitro transport study (FIG. 5C) showed that LbL coated liposomes assist the transport of insulin mainly via transcellular transport primarily due to two reasons. Firstly, no free chitosan was in the present formulation to interact with tight junction proteins because after the final step of coating, all the free polymers were removed by ultracentrifugation. Secondly, the chances of the top chitosan layer defoliating and then interacting with tight junctions to enhance localized paracellular transport of released insulin were very low as seen from the slow release, and slow defoliation, as gauged by the zeta potential (FIG. 4D).

In addition, it is not likely that the LbL coated liposome with a size of about 200 nm, is able to pass through the paracellular space, because the tight junctional space when fully opened by any enhancers is only 20 nm. TEER values first decreased 1 hour after treatment and subsequently increased from 2 to 4 hours, following the same pattern as HBSS control (buffer without any nanoparticles), suggesting that the tight junction was not involved to cause any paracellular transport of insulin. The transported insulin detected in the basal compartment was due to transcellular transport of the endocytosed insulin loaded LbL coated liposome. This observation was aligned with confocal analysis which further confirmed that the endocytosed particles entered the cytoplasm and travelled towards the basal side of the cell (FIG. 5A to FIG. 5D).

The amount of insulin transported across the Caco-2 monolayer was 3-fold higher when loaded in the LbL coated liposome compared to bare insulin solution. Bioactivity of the transported insulin can be directly measured by its ability to trigger glucose uptake in adipocytes. In type I diabetes, the body’s own immune cells destroy the insulin producing β cells, as a result glucose cannot enter the adipose or muscle cells for adenosine triphosphate (ATP) production. Bioactive insulin can bind to the insulin receptor on the adipocyte surface to initiate the entry of 2-deoxyglucose (2DG) and accumulation of deoxyglucose-6-phosphate (2DG6P) inside the cell, which can be converted to a luminescent signal for detection. In the present study, the transported insulin retained its bioactivity while crossing the Caco-2 monolayer as demonstrated by the glucose uptake by matured 3T3 L1-MBX adipocytes. This is encouraging because one of the most challenging problems for oral drug delivery is to prevent drug degradation and to retain bioactivity during GI transit and intestinal absorption.

A preliminary feasibility study in an animal model demonstrated that the lyophilized formulation assisted the absorption of insulin in rats when oral gavaged in solution form. Lyophilisation of the LbL coated liposome with 5% trehalose preserves the particle and ensures longer shelf life. Rapid absorption of the formulation results in a peak in plasma insulin 0.5 hour post oral gavaging at 3 mIU L⁻¹ (FIG. 8). In contrast, absorption of commercial insulin-loaded nanoparticles prepared using an emulsion technique, was prolonged with a broad peak of absorption between 6-8 hours. The study reported the absorption was due to mucoadhesion of the nanoparticles which gave out localized delivery of insulin on the small intestinal wall. Although similar levels of plasma insulin were detected (~5 mIU L⁻¹) with lower dose (50 IU kg⁻¹) of the commercial nanoparticles, it is worth noting that the plasma insulin peak was broad and was too close to that of the basal level to give a conclusive evidence of significant absorption. On the other hand, the present technology resulted in rapid absorption (0.5 hour) with a sharp peak in plasma insulin at about 3 µIU mL⁻¹ after oral administration of LbL-liposome with only 5 layers of insulin, and the duration in the blood stream was short (up till 4 hours) due to rapid elimination, demonstrating a proof of concept. This plasma insulin can be further increased just by repeating the number of alternating insulin and chitosan layers on the liposome surface. Theoretically, a total of 182 alternating layers (91 layers of insulin) could be coated on the liposomes’ surface while keeping the size of the particle at less than 500 nm for optimal cellular uptake. This brings significant clinical importance for the purpose of blood glucose reduction.

In another study, the serum insulin level peaked at about 50 mIU L⁻¹ 5 hours after the oral administration of the powdered form of TPP chitosan nanoparticles loaded in enteric capsules. From the stability study, these pH dependent nanoparticles aggregated at pH 7 and above rapidly releasing more than 60% of insulin within 4 hours at pH 7.4. The pH in the intestinal tract varied from pH 6.6 ± 0.5 in the proximal small intestine to pH 7.5 ± 0.4 in the terminal ileum. By the end of 3 hours when the capsule releases the formulation, it was expected to aggregate and lose its ability to enhance cellular uptake and transport, the only possible reason for the absorption was paracellular transport with the aid of disintegrated chitosan polymer from the destabilized formulation. Premature release of insulin in the GI tract may expose the protein in the enzyme-rich brush border environment of the small intestine, which indicates that the carrier was incapable of protecting its payload during intestinal penetration. Indeed, the TPP chitosan nanoparticle was not optimized at transcellular transport of nanoparticles across the intestinal epithelium, but functions as a permeation enhancer which was released due to particle instability in the intestinal pH to permit paracellular diffusion of insulin. A major drawback of using permeation enhancer is the potential damage to the intestinal lining. Major pharmaceutical companies focused on permeation enhancers, namely sodium caprate and ethylenediaminetetraacetic acid (EDTA), respectively. However, for potential treatment of a chronic metabolic disease whereby repeated once-daily administration is inevitable, prolonged exposure of the intestine to high local concentrations of sodium caprate or EDTA imposes unavoidable safety concerns. In addition, bioavailability of such drugs (having the permeation enhances) orally administered remained low, and it was hypothesized that the unabsorbed insulin might result in increased risk of proliferative effects (cancer-causing) in localized areas of the gastrointestinal tract due to direct exposure to high levels of such drugs with permeation enhances. Due to such safety concerns, long-term safety trials remain necessary to evaluate the possible outcomes of persistent exposure of the intestine to high local concentrations of permeation enhancers.

In comparison, the present technology delivers insulin via a transcellular pathway which is safer and more desirable because absorption take place without disrupting the tight junctions, which is advantageous in maintaining the barrier function. The present LbL coated liposome was able to protect insulin during intestinal penetration and the loading can be further improved by increasing the number of insulin layers. Considering the protein nature of insulin and its usual destiny after oral administration, the current finding is encouraging for further translation. In summary, these results (whether in vitro and in vivo) indicate the potential of the LbL technology using chitosan and insulin as an oral delivery system for treating diabetes mellitus.

### Example 4: A Non-Limiting Example of Coating Conditions

The present example demonstrates for a coating approach that confers a significantly higher loading of drugs in the present liposomes for oral applications. Particularly, the present coating conditions is easier for forming liposomes having more than 5 layers of coating with better stability for oral insulin application. Also, the present coating approach confers better size control and demonstrates feasibility through both detailed in vitro and in vivo studies. The present coating condition employs a different buffered condition (carbonate-bicarbonate buffer, pH 9.6) to dissolve insulin and the loading of insulin was significantly increased from 1.2% to 10.8% as the number of layers were increased from 5 to 11. It is to be noted insulin that may be present in the liposome core are not included in these loadings, i.e. the loadings refer to insulin in the layers coated on the liposome core. This is extremely promising because most of the existing nanomedicine suffers from low drug loading with almost all formulations, with the drug being less than 10% of the nanocarrier weight. This has traditionally been a stumbling block in translation, because even if sufficient absorption of nanocarriers occurs in the small intestine, drug bioavailability may still be inadequate for therapeutic effect.

The present coating approach does not suffer from a limitation of size controllability as the number of layers increased and confers better size control even if the protein possesses a pI of 7, which has a lower degree of ionization, over coating techniques involving the use of sodium phosphate dibasic pH 7.5 for coating the insulin layer. With this approach, coated particle exhibited increased stability and controllability over size when coating layers increase beyond 5. Carbonate-bicarbonate buffer makes the insulin highly ionized and negatively charged at pH 9.6 and therefore enable repeated cycles of coating with better size control. Theoretically, the current method of coating ensures repeated cycles of coating and the number of layers can go as high as possible. The method of coating in the present example improves the technology, as only through increasing the number of layers can higher loading and higher bioavailability be achieved for its application in oral insulin.

Previously coated nanoparticle was stable in solution, in the current example, the feasibility of converting the formulation from solution to powdered form which significantly increases the shelf-life is demonstrated. Using the present coating method, the particle size characterized using DLS reflected excellent controllability over size when the number of layers increase to 11. This improvement in coating method enables large amount of insulin to be loaded on the liposome surface, which in turn increases the amount of insulin crossing the intestinal epithelial cells. With the present method, a peak in plasma insulin in a group of 4 wistar rats 0.5 hour post oral gavaging can be observed. Furthermore, bioactivity of the 11 layers coated LbL coated liposome using 3T3 L1-MBX adipocytes was investigated. The ability of the transported insulin to trigger glucose uptake in these adipocytes showed that the insulin retained its bioactivity while crossing the intestinal epithelial cell barrier. The carrier was able to give intracellular protection against lysosomal degradation and ensure the payload to reach the blood stream safely. This is especially encouraging considering the protein nature of insulin, to overcome the barriers and get detected in the systemic circulations means the carrier was able to protect large amount of insulin against the GI environment, helping them to cross the absorption barrier to reach the blood. The present method of coating offers a new platform for further increase the number of layers to increase the loading, because of the new buffer condition used, the number of layers could increase with excellent size controllability.

The present coating method was demonstrated through both in vitro and in vivo studies and the formulation from the present coating method has additional features including higher drug loading, protection against GI environment (almost no release in SIF pH 6.8 for 5 weeks, and penetration of intestinal epithelium with retention of bioactivity. Such advantages has a considerable impact in determining the feasibility and effectiveness of an oral insulin formulation using LbL coated liposome as a carrier.

The coating method of the present example involves changing of coating condition for better size control and higher number of layers. The present coating method involved the use of carbonate-bicarbonate buffer pH 9.6 instead of sodium phosphate bibasic pH 7.4 to dissolve insulin. This buffer condition confers better size control when the number of coating layers increase beyond 5. Thus, more insulin gets loaded (see FIG. 3B) and only with more insulin, better transport and absorption are achieved for application in oral insulin delivery.

The coating method of the present example is feasible for a wider range of application. For instance, the present coating method can be extended to 3 different molecular weights of chitosan including chitosan 15 kDa, chitosan 190-50 kDa, chitosan 310-190 kDa (see FIGS. 2A and 2B). Furthermore, bovine serum albumin was also successfully coated using the same technique, proving the coating technique to be effective regardless of the molar mass of the polymer or protein used.

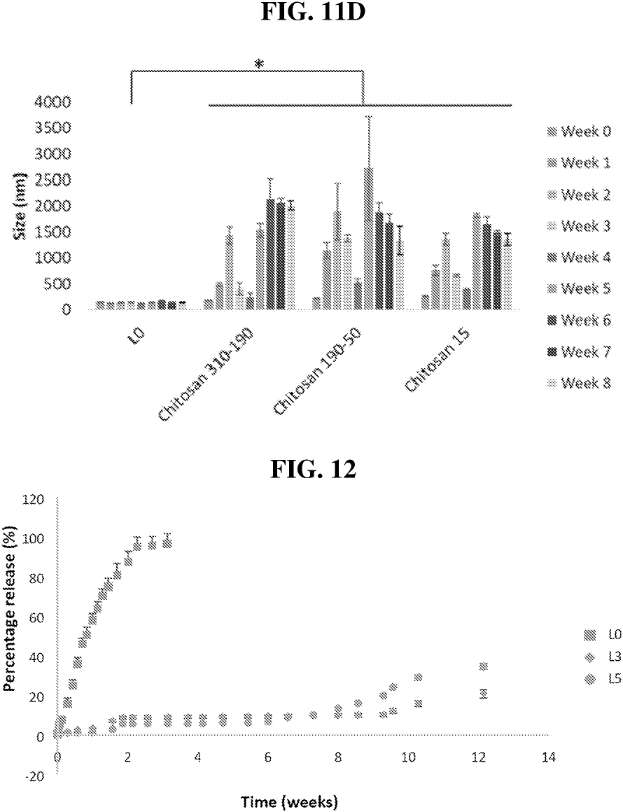

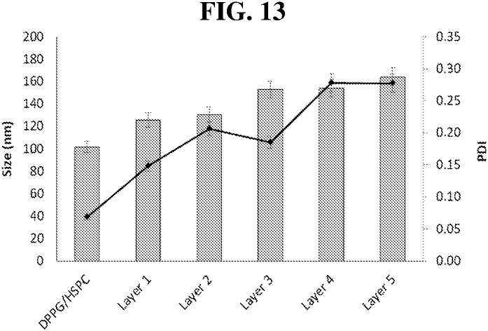

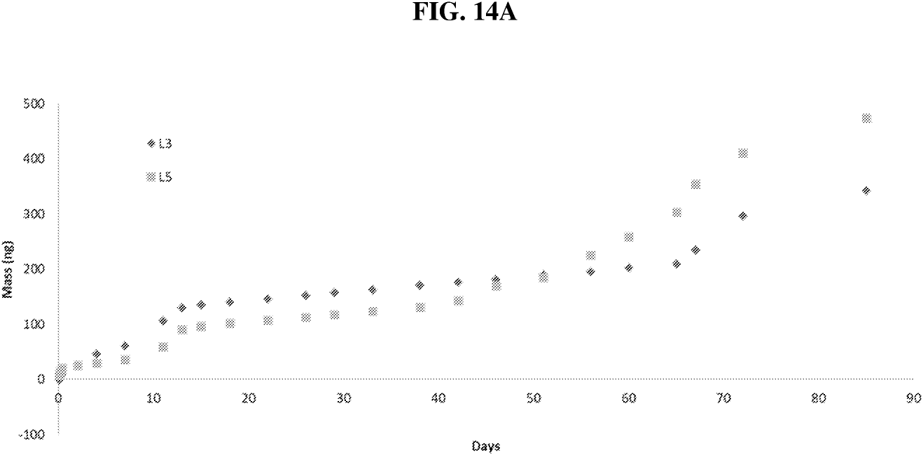

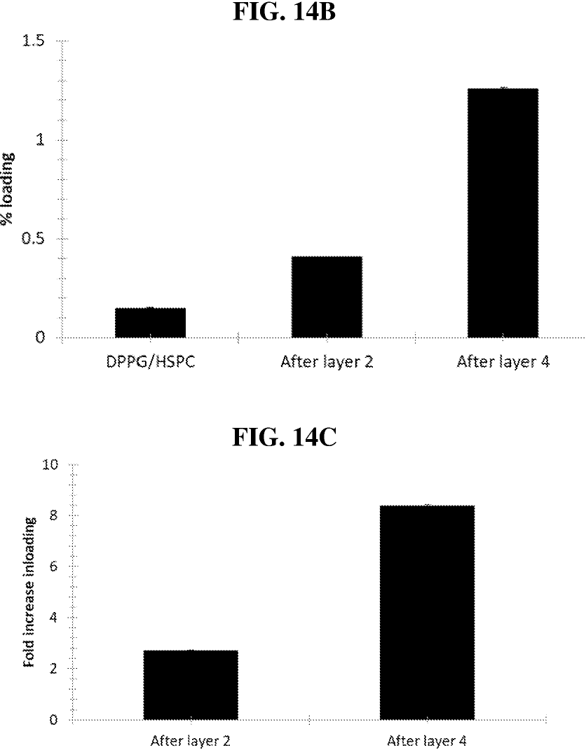

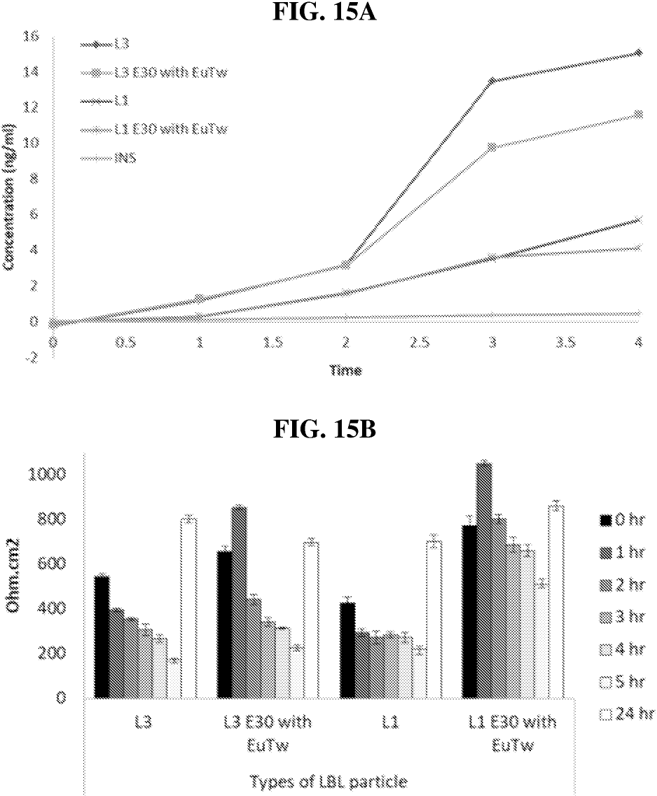

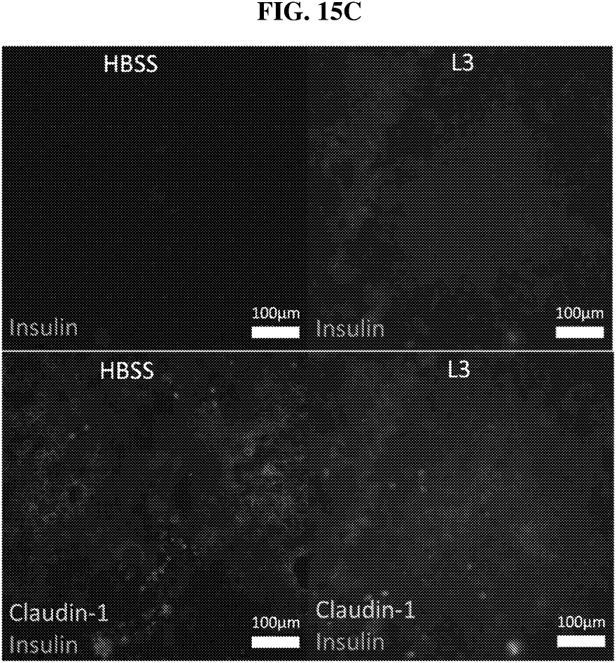

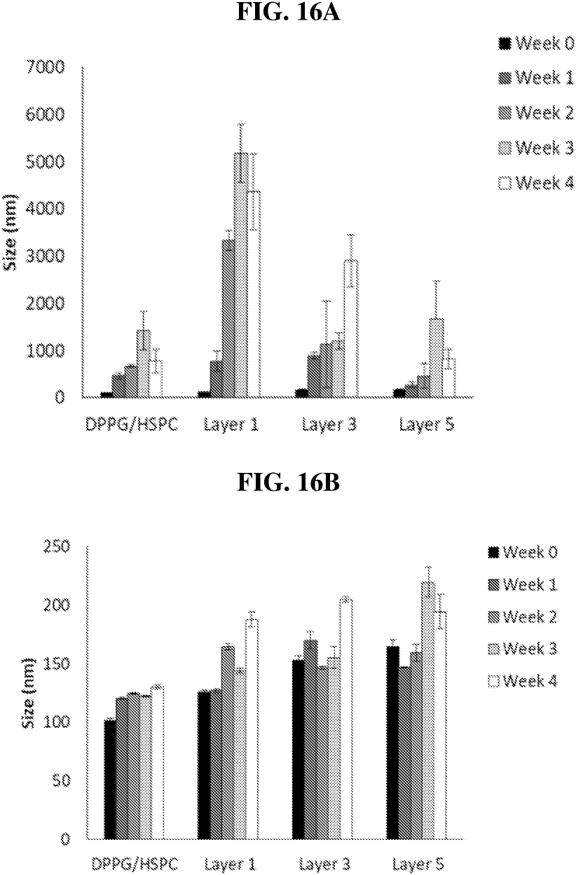

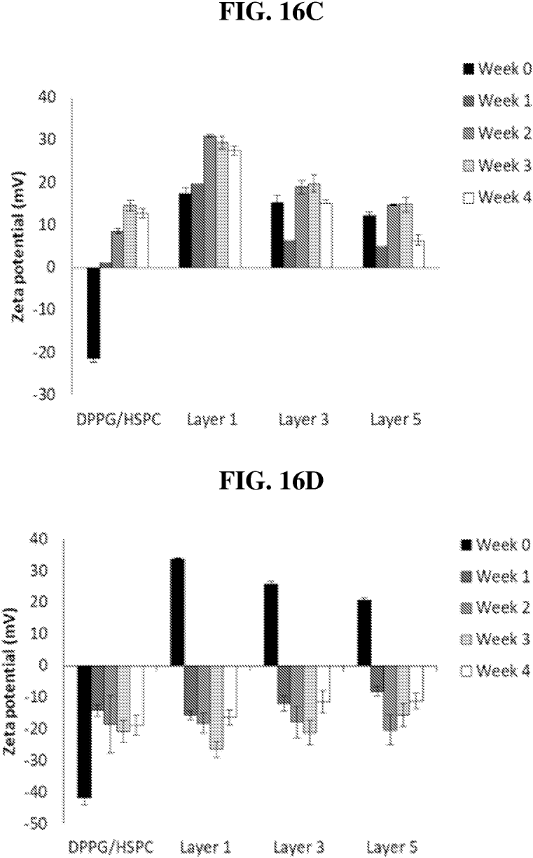

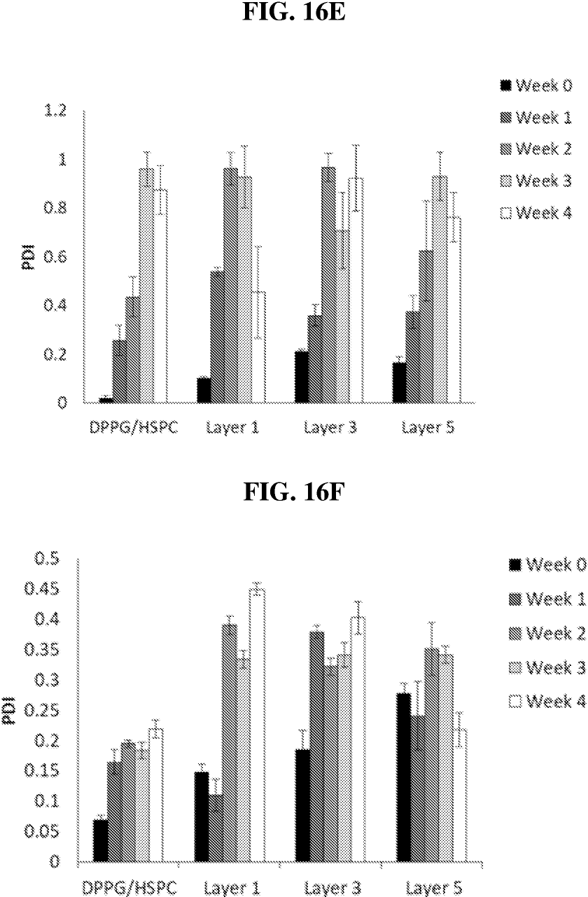

The coating method of the present example has ability to protect against gastrointestinal environment. The present coating method affords 11 layers of LbL coated liposome which demonstrated its ability to protect the insulin payload during its transit in GI tract as shown by its sustained release in simulated intestinal environment pH 6.8 (see FIG. 11B).

The coating method of the present example confers higher loading of drugs, e.g. insulin. The present coating method enables much higher loading of insulin (10.8% by weight) with 11 layers of coating, which can be 9-fold higher than reported methods (see FIG. 3B).

The coating method of the present example is capable of apical to basal intracellular transport. The present coating method demonstrated cellular uptake using fluorescently tagged lipid bilayer in the liposome, which shows intracellular trafficking of the LbL coated liposome from apical to basal lateral side of the Caco-2 monolayer (see FIGS. 5A to 5D).

The present coating method allows for a solution containing the present multilayered liposomes to be converted to a powdered form for extended shelf-life (stability). The present coating method extended stability of these LBL by lyophilization technique, using cryoprotectant trehalose, the 11 layers of LbL coated liposomal formulation was able to be converted into powdered form for longer storage.

The present coating method does not compromise, but maintains the bioactivity of the drug in the present multilayered liposome. A bioactivity study of transported insulin was conducted, which was demonstrated using matured 3T3 L1-MBX adipocytes. This is encouraging because one of the most challenging problems for oral drug delivery is to prevent drug degradation and to retain bioactivity during GI transit and intestinal absorption.

The present coating method has been demonstrated for in vivo absorption in rat. The present coating method demonstrated in vivo feasibility through pharmacokinetic study in rats. Oral administration of these LbL coated liposomes in rats showed a peak in plasma insulin post oral gavaging the reconstituted lyophilized formulation (see FIG. 8).

The present coating method confers coating of more than 5 layers. FIGS. 9B and 13 depict for coating insulin onto a liposome surface for up to 5 layers using sodium phosphate dibasic as a medium, as there was certain difficulty forming beyond 5 layers because of the loss of control of size as the number of coating layer increases. Moreover, little or no apical to basal intracellular trafficking was observed when demonstrating for transport and cellular uptake using a 3-layer of LbL coated liposome.

### Example 5: Commercial and Potential Applications

The present disclosure provides for a layer by layer technique of surface modifying liposomes with alternating layers of chitosan and insulin, leading to a liposome-based nanocarrier with high insulin loading (10% or more by weight). The LbL coated liposome demonstrated excellent stability for a 4 weeks study in PBS pH 7.4 at 37° C. The outermost chitosan layer of the LbL coated liposome facilitated cellular uptake and transport by Caco-2 cells and the transported insulin demonstrated retention of bioactivity through glucose uptake assay performed on 3T3 L1-MBX adipocytes. These LbL coated liposomes were able to protect insulin during its GI transit and ensure its insulin payload reached the systemic blood circulation, as verified in a pharmacokinetic study in a rat model, thus indicating the potential application of these nanoparticles in the field of oral protein delivery.

The present disclosure also identifies differences between the present multilayered liposome, its method of coating and those traditionally developed. There are a number of features that distinguish the present disclosure. For example, the present coating methods/conditions and the resultant liposomal formulation exhibited better drug loading, protection against GI environment, and penetration of intestinal epithelium with retention of bioactivity. The present technology provides a distinguished approach to coat the particles with high drug loading, which was able to overcome the GI barriers, cross the intestinal epithelium, and eventually reach the blood circulation. The present technology is versatile, i.e. other molar masses of polymer coating or protein drug can be used, conferring a wider application. The list of characteristics of the present liposome is tabulated in Table 1 below.

TABLE 1

Characteristics of Liposome of Present Disclosure

| | Characteristics | Present Disclosure |
|---|---|---|
| 1 | Composition, range of application, and coating conditions | Protein: insulin dissolved in Carbonate-Bicarbonate buffer Polymer: chitosan 15 kDa, chitosan 190-50 kDa, chitosan 350-190 kDa, |
| 2 | Number of layers | 11 layers and could potentially be increased further because of the new coating buffer condition (FIGS. 2A and 2B) |
| 3 | Protection against GI environment (SIF release) | Almost no release in the first 5 weeks in SIF pH 6.8, indicating the ability to protect insulin from being exposed to GI environment in the small intestine. (FIG. 11A) |
| 4 | Loading | High loading for nanocarrier (10.8% by weight) (FIG. 3A), almost 9 fold higher than reported example |
| 5 | Apical to basal intracellular trafficking | Plane by plane detailed analysis by confocal microscopy revealed apical to basal intracellular trafficking of the internalized LbL liposomes (FIG. 5A) |
| 6 | Stability | Lyophilisation condition can convert the solution into powdered form for extended storage (FIG. 7 ) |
| 7 | Retention of bioactivity | Transported insulin demonstrated retention of bioactivity while crossing intestinal epithelial cells (FIGS. 6A to 6F) |
| 8 | In vivo study | Feeding rats with reconstituted formulation resulted in rapid absorption 0.5 hour post oral gavaging (FIG. 8 ) |

While the present disclosure has been particularly shown and described with reference to specific embodiments, it should be understood by those skilled in the art that various changes in form and detail may be made therein without departing from the spirit and scope of the present disclosure as defined by the appended claims. The scope of the present disclosure is thus indicated by the appended claims and all changes which come within the meaning and range of equivalency of the claims are therefore intended to be embraced.

1. A multilayered liposome comprising:
a liposome core defined by a lipid layer; and
five or more coating layers surrounding the lipid layer,
wherein the five or more coating layers comprise more than one positively charged polymeric layer and more than one negatively charged drug layer,
wherein the more than one positively charged polymeric layer and the more than one negatively charged drug layer are deposited in an alternating manner,
wherein one of the more than one positively charged polymeric layer is formed as an outermost coating layer, and
wherein each of the more than one negatively charged drug layer comprises insulin, an insulin-like factor, a growth factor, or a hormonal peptide.

2. (canceled)

3. (canceled)

4. The multilayered liposome of claim 1, wherein
the lipid layer is negatively charged and comprises hydrogenated soybean phosphatidylcholine and 1,2-dipalmitoyl-sn-glycero-3-phosphoglycerol in a molar ratio of 10:1; or
the lipid layer is positively charged and comprises hydrogenated soybean phosphatidylcholine and 1,2-dioleoyl-3-trimethylammoniumpropane in a molar ratio of 10:1.

5. The multilayered liposome of claim 1, wherein each of the more than one positively charged polymeric layers comprises chitosan, poly L-arginine, poly L-lysine, polyallylamine hydrochloride, polyethylenimine, or polyamidoamine.

6. (canceled)

7. The multilayered liposome of claim 1, wherein the liposome core comprises a drug, wherein the drug comprises insulin, an insulin-like factor, a growth factor, or a hormonal peptide.

8. The multilayered liposome of claim 1, wherein the more than one negatively charged drug layer has a drug loading of at least 1 wt%.

9. The multilayered liposome of claim 1,
wherein the five or more coating layers comprise eleven coating layers, and
wherein the more than one negatively charged drug layer has a drug loading of at least 10 wt%.

10. The multilayered liposome according to claim 1, further comprising an enteric coating layer formed outer to the outermost coating layer, wherein the enteric coating layer swells minimally at a pH ranging from 1 to 2.

11. (canceled)

12. (canceled)

13. A method of treating diabetes mellitus, the method comprising orally administering the multilayered liposome of claim 1.

14. A method of producing the multilayered liposome of claim 1, the method comprising:
providing liposomes each having a liposome core defined by a lipid layer;
forming one negatively charged drug layer or one positively charged polymeric layer on the liposome core;
depositing one positively charged polymeric layer on the formed negatively charged drug layer or one negatively charged drug layer on the formed positively charged polymeric layer;
repeating the deposition of one negatively charged drug layer on the positively charged polymeric layer earlier deposited or one positively charged polymeric layer on the negatively charged drug layer earlier deposited so as to have
(i) five or more coating layers surrounding the lipid layer, and
(ii) the more than one positively charged polymeric layer and the more than one negatively charged drug layer deposited in an alternating manner,
wherein one of the more than one positively charged polymeric layer is formed as an outermost coating layer, and
wherein each of the more than one negatively charged drug layer comprises insulin, an insulin-like factor, a growth factor, or a hormonal peptide.

15. The method of claim 14, wherein providing the liposomes comprises:
forming a thin film from a solution comprising one or more lipids; and
contacting the thin film with one or more drug solutions in a stepwise manner, wherein the contacting of the thin film with each drug solution is carried out after a time interval from another.

16. The method of claim 14, wherein the liposomes are diluted in a carbonate-bicarbonate buffer prior to forming one negatively charged drug layer or one positively charged polymeric layer on the liposome core.

17. The method of claim 14, wherein forming one negatively charged drug layer on the liposome core comprises:
mixing a carbonate-bicarbonate buffer comprising a drug with the liposomes to form a first mixture; and
centrifuging the mixture to obtain liposomes having the negatively charged drug layer formed thereon.

18. The method of claim 17, wherein depositing one positively charged polymeric layer on the formed negatively charged drug layer comprises:
mixing an organic acid comprising a polymer with the liposomes having the negatively charged drug layer formed thereon to form a second mixture; and
centrifuging the mixture to obtain liposomes having the positively charged polymeric layer deposited thereon.

19. The method of claim 14, wherein forming one positively charged polymeric layer on the liposome core comprises:
mixing an organic acid comprising a polymer with the liposomes to form a first mixture; and
centrifuging the mixture to obtain liposomes having the positively charged polymeric layer formed thereon.

20. The method of claim 19, wherein depositing one negatively charged drug layer on the formed positively charged polymeric layer comprises:
mixing a carbonate-bicarbonate buffer comprising a drug with the liposomes having the positively charged polymeric layer formed thereon to form a second mixture; and
centrifuging the mixture to obtain liposomes having the negatively charged drug layer deposited thereon.

21. A multilayered liposome comprising:
a liposome core defined by a lipid layer;
five or more coating layers surrounding the lipid layer;
an outermost coating layer which is positively charged;
wherein the five or more coating layers comprise more than one negatively charged polymeric layer and more than one positively charged drug layer,
wherein the more than one negatively charged polymeric layer and the more than one positively charged drug layer are deposited in an alternating manner; and
wherein each of the more than one positively charged drug layer comprises insulin, an insulin-like factor, a growth factor, or a hormonal peptide.

22. The multilayered liposome of claim 21,
wherein each of the more than one negatively charged polymeric layers comprises a polymer having a —COOH functional group or a —COO⁻ functional group,
wherein the polymer having the —COOH functional group or the —COO⁻ functional group comprises hyaluronic acid, sodium alginate, or a copolymer derived from methacrylic acid, methyl acrylate and/or methyl methacrylate.

23. The multilayered liposome of claim 21, wherein the outermost coating layer comprises a positively charged polymeric layer, wherein the positively charged polymeric layer comprises chitosan, poly L-arginine, poly L-lysine, polyallylamine hydrochloride, polyethylenimine, or polyamidoamine.

24. The multilayered liposome according to claim 21, further comprising an enteric coating layer formed outer to the outermost coating layer, wherein the enteric coating layer swells minimally at a pH ranging from 1 to 2.

25. A method of producing the multilayered liposome of claim 21, the method comprising:
providing liposomes each having a liposome core defined by a lipid layer;
forming one positively charged drug layer or one negatively charged polymeric layer on the liposome core;
depositing one negatively charged polymeric layer on the formed positively charged drug layer or one positively charged drug layer on the formed negatively charged polymeric layer;
repeating the deposition of one positively charged drug layer on the negatively charged polymeric layer earlier deposited or one negatively charged polymeric layer on the positively charged drug layer earlier deposited so as to have
(i) five or more coating layers surrounding the lipid layer, and
(ii) the more than one negatively charged polymeric layer and the more than one positively charged drug layer deposited in an alternating manner,
forming an outermost coating layer which is positively charged, and
wherein each of the more than one positively charged drug layer comprises insulin, an insulin-like factor, a growth factor, or a hormonal peptide.
